(19) United States
(12) Patent Application Publication
(10) Pub. No.: US 2024/0033233 A1
(43) Pub. Date: Feb. 1, 2024

(54) COMPOSITIONS AND METHODS FOR DELIVERING PHARMACEUTICAL AGENTS

(71) Applicant: Arizona Board of Regents on Behalf of the University of Arizona, Tucson, AZ (US)

(72) Inventors: Heidi Mansour, Tucson, AZ (US); Victor Ruiz, Tucson, AZ (US); Sally E. Dickinson, Tucson, AZ (US); Clara N. Curiel, Tucson, AZ (US); Hsiao-Hui Chow, Tucson, AZ (US); Georg T. Wondrak, Tucson, AZ (US)

(21) Appl. No.: 18/018,924

(22) PCT Filed: Jul. 30, 2021
(86) PCT No.: PCT/US2021/043829

§ 371 Date: Jan. 31, 2023

Related U.S. Application Data
(60) Provisional application No. 63/059,274, filed on Jul. 31, 2020.

(51) Int. Cl. A61K 31/18; A61K 47/10; A61K 9/00; A61K 9/06

(52) U.S. Cl. CPC A61K 31/18; A61K 47/10; A61K 9/0014; A61K 9/06

(57) ABSTRACT

Provided herein are compositions and methods for delivering pharmaceutical agents. In particular, provided herein are topical formulations of resatorvid for use in treating and preventing skin pathologies.

### CROSS-REFERENCE TO RELATED APPLICATION

This application claims priority to and the benefit of U.S. Provisional Application No. 63/059,274, filed Jul. 31, 2020, which is hereby incorporated by reference in its entirety.

### STATEMENT REGARDING FEDERALLY SPONSORED RESEARCH OR DEVELOPMENT

This invention was made with government support under Grant No. P01 CA229112, awarded by National Institutes of Health. The government has certain rights in the invention.

### FIELD

Provided herein are compositions and methods for delivering pharmaceutical agents. In particular, provided herein are topical formulations of resatorvid for use in treating and preventing skin pathologies.

### BACKGROUND

Skin cancers tend to be slow growing and can be cured with early and adequate treatment. Non-melanoma skin cancer is the most common neoplasm in countries with high rates in Caucasian populations and in cases of high UV ray exposure (Lomas et al., British Association of Dermatologists 2012 166, pp1069-1080). In the United States alone, there are more than half a million new cases per year. Despite the fatality rate of Non-melanoma skin cancer being minimal, it is known to cause significant impact including severe local destruction, permanent disfigurement and disability.

Currently, there are several recommended preventive measures for skin cancer including recommendations for avoiding UV rays from the sun or tannings beds, utilizing broad spectrum sunscreen and scheduling routine dermatologist examinations. Even with these preventive measures available to most, skin cancers cases are still bountiful. There is a need for more advanced preventive measures that would significantly reduce the amount of cases yearly. In addition, there is a need for additional treatments for skin cancers and other skin pathologies.

### SUMMARY

Provided herein is a formulation of resatorvid, which is an investigational fluorinated small molecular weight drug, as well as its processing/manufacture and use. This formulation is a selective TLR4 inhibitor and is hydrophobic. Existing formulations of resatorvid are not soluble and are unable to penetrate the skin. Provided herein are drug delivery formulations of resatorvid that utilize FDA approved excipients that are biocompatible with skin. The formulation can be made into serums/hydrogels, creams and lotions. In order to develop formulations suitable for topical delivery and penetration through the skin, it was necessary to test many formulations and develop specific methods for compounding resatorvid into a stable, usable formulation. The compositions provide treatments and preventative agents, for example, for a variety of skin pathologies.

For example, in some embodiments, provided herein is a composition, comprising: a pharmaceutical composition comprising resatorvid (e.g., a racemic mixture or an enantiomer based on its stereochemistry) and a pharmaceutically acceptable carrier, wherein the composition is formulated for topical delivery. In some embodiments, the composition inhibits TLR4 in the skin.

The present disclosure is not limited to a particular formulation. Exemplary formulations comprise hydrophobic carriers (e.g., pharmaceutically acceptable carriers. Examples include but are not limited to, a cream, a liquid (e.g., solution), a hydrogel, a serum, or a lotion.

In some embodiments, the cream and/or lotion comprises an emollient (e.g., comprising one or more of petrolatum, lanolin, mineral oil, dimethicone, glycerin, lecithin, or propylene glycol). In some embodiments, the cream or lotion further comprises polyethylene glycol (PEG) United States Pharmacopeia (USP) (e.g., PEG 400 USP or PEG 600 USP) or propylene glycol USP (PG USP), for example, at a concentration of 0.1 to 2 ml per 1 gram of cream. In some embodiments, compositions are incorporated into a drug delivery or micro needle drug delivery skin patch.

In some embodiments, liquid formulations comprise PEG USP (e.g., PEG 400 USP or PEG 600 USP) or PG USP as a solvent. The present disclosure is not limited to particular hydrogels. Examples include but are not limited to, carbomer, poloxamer, and/or hyaluronate, containing dissolved resatorvid in PEG USP or PG USP. In some embodiments, resatorvid is present in the pharmaceutical composition at a concentration of 0.01% to 10% w/v or % w/w (e.g., 0.05% to 5.0%, 0.1 to 5%, or 0.5 to 5%).

In certain embodiments, the composition is prepared by a method, comprising: a) dissolving resatorvid in a liquid selected from PEG-600 or PEG-400 using sonication to generate a resatorvid solution; and b) mixing the resatorvid solution and the pharmaceutically acceptable carrier to generate the composition.

Further embodiments provide a method of treating or preventing a skin pathology, comprising: topically administering a composition described herein to a subject in need thereof.

Additional embodiments provide a method of inhibiting TLR4 in the skin of a subject (e.g., to treat or prevent a skin pathology), comprising: topically administering a composition described herein to a subject in need thereof.

Yet other embodiments provide use of a composition described herein to treat or prevent a skin pathology in a subject or a composition as described herein for use in treating or preventing a skin pathology in a subject.

Certain embodiments provide use of a composition described herein to inhibit TLR4 in a subject or a composition as described herein for use in inhibiting TLR4 in a subject.

The present disclosure is not limited to particular skin pathologies. Examples include but are not limited to, pathologies related to exposure to environmental stressors, photoaging and chronological aging, inflammatory dysregulation, microbial infection, or skin malignancies and premalignancies. In some embodiments, the environmental stressor is selected from, for example, solar radiation (e.g., ultraviolet, visible, or infrared), ionizing radiation, pollutants (e.g., dioxin, benzpyrene, particulate matter, or arsenic.), or natural or artificial allergens.

In some embodiments, the inflammatory dysregulation is, for example, erythema, urticaria, telangiectasia, edema, impaired skin barrier function, atopic dermatitis, contact dermatitis, eczematous dermatitis, allergic reactions, autoimmune conditions (e.g., lupus or psoriasis), scleroderma, or wounds (e.g., diabetic wounds, burn wounds, or radiation-induced injury wounds). In some embodiments, the microbial infection is a fungal, bacterial, or viral pathogen.

The present disclosure is not limited to particular skin malignancies and premalignancies. Examples include but are not limited to, melanoma, nonmelanoma skin cancer, actinic keratosis, or dysplastic nevi.

Additional embodiments are described herein.

### DESCRIPTION OF THE FIGURES

FIG. 1 shows differential scanning calorimetry of resatorvid.

FIG. 2 shows hot stage microscopy of resatorvid under cross-polarizers.

FIG. 3 shows X-ray powder diffraction of resatorvid.

FIG. 4 shows HPLC analysis of resatorvid. A) calibration curve on an HPLC column of 150 mm in length. B) chromatogram of a single run of resatorvid on an HPLC column of 150 mm in length. C) chromatogram of multiple concentrations of resatorvid on an HPLC column of 150 mm in length. D) chromatogram of a single run of resatorvid on an HPLC column of 250 mm in length. E) chromatogram of a single run of resatorvid on an HPLC column of 250 mm in length. F) chromatogram of multiple concentrations of resatorvid on an HPLC column of 250 mm in length. G) calibration curve on an HPLC column of 250 mm in length.

FIG. 5 shows SEM micrographs of Resatorvid (TAK-242) at 400×, 1300×, 3000× and 5000× resolution.

FIG. 6 shows EDX spectra of raw Resatorvid (TAK-242) powder showing characteristic peaks. C—Carbon, O—Oxygen, S—Sulfur, F—Fluorine, and Cl—Chlorine atoms.

FIG. 7 shows Representative Raman spectra of raw Resatorvid (TAK-242) using 785 nm laser.

FIG. 8 shows a DSC thermogram of raw Resatorvid (TAK-242).

FIG. 9 shows an ATR-FTIR spectrum of raw Resatorvid (TAK-242).

FIG. 10 shows a UV scan of 0.1% w/v Resatorvid (TAK-242) in HPLC grade methanol being compared to HPLC methanol alone at range of 200 nm to 700 nm.

FIG. 11 shows Strat-M® permeation profile of Resatorvid (TAK-242) 5% w/w DermaBase (cream formulation).

FIG. 12 shows Strat-M® permeation profile of Resatorvid (TAK-242) 5% w/w PENcream (cream formulation).

FIG. 13 shows Strat-M® permeation profile of Resatorvid (TAK-242) 5% w/w DermaBASE (lotion formulation).

FIG. 14 shows Strat-M® permeation profile of Resatorvid (TAK-242) 5% w/v polyethylene glycol 400 (simple solution).

FIG. 15 shows Strat-M® permeation profile of Resatorvid (TAK-242) 5% w/v propylene glycol (simple solution).

FIG. 16 shows a comparison of Strat-M® permeation profiles of Resatorvid (TAK-242) 5% creams and lotion formulations.

FIG. 17 shows a comparison of Strat-M® permeation profiles of Resatorvid (TAK-242) 5% simple solutions.

FIG. 18 shows Strat-M® permeation profile of Resatorvid (TAK-242) 2.5% w/w DermaBASE (cream formulation)

FIG. 19 shows Strat-M® permeation profile of Resatorvid (TAK-242) 2.5% w/w PENcream (cream formulation).

FIG. 20 shows Strat-M® permeation profile of Resatorvid (TAK-242) 2.5% w/w DermaBASE (lotion formulation).

FIG. 21 shows Strat-M® permeation profile of Resatorvid (TAK-242) 2.5% w/v polyethylene glycol 400 (simple solution).

FIG. 22 shows Strat-M® permeation profile of Resatorvid (TAK-242) 2.5% w/v propylene glycol (simple solution).

FIG. 23 shows Strat-M® permeation profile of Resatorvid (TAK-242) 2.5% w/w Carbomer 934P resin (gel formulation).

FIG. 24 shows Strat-M® permeation profile of Resatorvid (TAK-242) 2.5% w/w Sodium hyaluronate (serum/gel formulation).

FIG. 25 shows Strat-M® permeation profile of Resatorvid (TAK-242) 2.5% w/v Pluronic F127 (gel formulation).

FIG. 26 shows a comparison of Strat-M® permeation profiles of Resatorvid (TAK-242) 2.5% creams and lotion formulations.

FIG. 27 shows a comparison of Strat-M® permeation profiles of Resatorvid (TAK-242) 2.5% simple solutions.

FIG. 28 shows a comparison of Strat-M® permeation profiles of Resatorvid (TAK-242) 2.5% gels and serum solutions.

FIG. 29 shows Strat-M® permeation profile of Resatorvid (TAK-242) 1.25% w/w DermaBASE (cream formulation).

FIG. 30 shows Strat-M® permeation profile of Resatorvid (TAK-242) 1.25% w/w PENcream (cream formulation).

FIG. 31 shows Strat-M® permeation profile of Resatorvid (TAK-242) 1.25% w/w DermaBASE (lotion formulation).

FIG. 32 shows Strat-M® permeation profile of Resatorvid (TAK-242) 1.25% w/w polyethylene glycol 400 (simple solution).

FIG. 33 shows Strat-M® permeation profile of Resatorvid (TAK-242) 1.25% w/w propylene glycol (simple solution).

FIG. 34 shows Strat-M® permeation profile of Resatorvid (TAK-242) 1.25% w/w Carbomer 934P resin (gel formulation).

FIG. 35 shows Strat-M® permeation profile of Resatorvid (TAK-242) 1.25% w/w Sodium hyaluronate (gel/serum formulation).

FIG. 36 shows Strat-M® permeation profile of Resatorvid (TAK-242) 1.25% w/v Pluronic F127 (gel formulation).

FIG. 37 shows a comparison of Strat-M® permeation profiles of Resatorvid (TAK-242) 1.25% creams and lotion formulations.

FIG. 38 shows a comparison of Strat-M® permeation profiles of Resatorvid (TAK-242) 1.25% simple solutions.

FIG. 39 shows a comparison of Strat-M® permeation profiles of Resatorvid (TAK-242) 1.25% gels and serum.

FIG. 40 shows in vitro Cell Viability of raw Resatorvid (TAK-242) using human transformed keratinocytes (HaCaT) and primary normal epidermal keratinocytes (NKEK).

FIG. 41 shows EpiDerm™ permeation profile of Resatorvid (TAK-242) 1.25% w/w DermaBASE (cream formulation).

FIG. 42 shows EpiDerm™ permeation profile of Resatorvid (TAK-242) 1.25% w/w PENcream (cream formulation).

FIG. 43 shows EpiDerm™ permeation profile of Resatorvid (TAK-242) 1.25% w/w DermaBASE (lotion formulation).

FIG. 44 shows EpiDerm™ permeation profile of Resatorvid (TAK-242) 1.25% w/v polyethylene glycol 400 (simple solution).

FIG. 45 shows EpiDerm™ permeation profile of Resatorvid (TAK-242) 1.25% w/v propylene glycol (simple solution).

FIG. 46 shows EpiDerm™ permeation profile of Resatorvid (TAK-242) 1.25% w/w Carbomer 934P resin (gel formulation).

FIG. 47 shows EpiDerm™ permeation profile of Resatorvid (TAK-242) 1.25% w/w Sodium hyaluronate (gel/serum formulation).

FIG. 48 shows EpiDerm™ permeation profile of Resatorvid (TAK-242) 1.25% w/v Pluronic F127 (gel formulation).

FIG. 49 shows a comparison of EpiDerm™ permeation profiles of Resatorvid (TAK-242) 1.25% creams and lotion formulations.

FIG. 50 shows a comparison of EpiDerm™ permeation profiles of Resatorvid (TAK-242) 1.25% simple solutions.

FIG. 51 shows a comparison of EpiDerm™ permeation profiles of Resatorvid (TAK-242) 1.25% gels and serum.

FIG. 52 shows an experimental protocol for in vivo treatment of mice with Resatorvid.

FIG. 53 shows results of pretreatment of mice with Resatorvid followed by SSL.

FIG. 54 shows results of pretreatment and posttreatment of mice with Resatorvid before and after SSL.

FIG. 55 shows results of pretreatment and posttreatment of mice with Resatorvid before and after SSL.

FIG. 56A-C shows results of pretreatment and posttreatment of mice with Resatorvid before and after SSL.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein, the term “diagnosed,” as used herein, refers to the recognition of a disease by its signs and symptoms (e.g., resistance to conventional therapies), or genetic analysis, pathological analysis, histological analysis, and the like.

As used herein, the term “effective amount” refers to the amount of a compound (e.g., a compound of the present disclosure) sufficient to effect beneficial or desired results. An effective amount can be administered in one or more administrations, applications or dosages and is not limited to a particular formulation or administration route.

As used herein, the term “co-administration” refers to the administration of at least two agent(s) (e.g., a compound of the present disclosure) and one or more additional agents or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA, (1975)).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, at least 65% free, at least 70% free, at least 75% free, at least 80% free, at least 85% free, at least 90% free, at least 95% free, at least 96% free, at least 97% free, at least 98% free, at least 99% free, or 100% free from other components with which they usually associated.

As used herein, the term “modulate” refers to the activity of a compound (e.g., a compound of the present disclosure) to affect (e.g., to promote or retard) an aspect of cellular function.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, condition, or injury is prevalent.

### DETAILED DESCRIPTION

Resatorvid (TAK-242) is a fluorinated investigational drug that is a TLR4 inhibitor. It has a chemical formula of C₁₅H₁₇NO₄ClFS

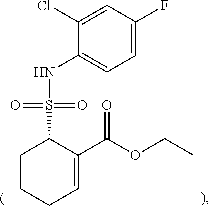 [SMILES: CCOC(=O)C1=CCCC[C@@H]1S(=O)(=O)Nc1ccc(F)cc1Cl]

molecular weight of 361.81 g/mol, cLogP is 3.69, reported Log P of 2.53, and a reported solubility of 20 mg/mL in EtOH, 10 mg/mL in DMSO, and is sparingly soluble in H2O. Resatorvid is expected to be hydrophobic.

Resatorvid was in clinical trials for sepsis and has been shown to be neuroprotective in TBI, ovarian and breast cancers, ALI, and rheumatoid arthritis. Initial studies (Blohm-Mangone et al., Cancer Prey Res; 11(5) May 2018; Janda et al., Photochemistry and Photobiology, 2016, 92: 816-825) indicated that an acetone solution of resatorvid blocked UV-induced AP-1 activation in mouse epidermis. However, such formulations are not suitable for use in human applications due to the acetone. The present disclosure provides topical formulations suitable for human use in treating and preventing a wide variety of skin pathologies.

Accordingly, in some embodiments, provided herein is a composition, comprising: a pharmaceutical composition comprising resatorvid (e.g., S enantiomer, R enantiomer, various proportions of R and S enantiomers, or a racemic mixture of 50% S and 50% R enantiomer), wherein the composition is formulated for topical delivery. In some embodiments, the composition inhibits TLR4 in the skin.

The present disclosure is not limited to a particular formulation. Exemplary formulations comprise hydrophobic carriers (e.g., pharmaceutically acceptable carriers). Examples include but are not limited to, a cream, a liquid, a hydrogel, or a lotion. Table 1 below shows exemplary formulations.

In some embodiments, the cream and/or lotion comprises an emollient (e.g., comprising one or more of petrolatum, lanolin, mineral oil, dimethicone, glycerin, lecithin, or propylene glycol USP). In some embodiments, commercially available emollients are used (e.g., PenCream or DermaCream).

In some embodiments, the cream or lotion further comprises polyethylene glycol (PEG) USP (e.g., PEG 400 USP or PEG 600 USP), for example, at a concentration of 01 to 2 ml per 1 gram of cream.

In some embodiments, liquid formulations comprise PEG USP (e.g., PEG 400 USP) or propylene glycol (PG) USP as a solvent.

The present disclosure is not limited to particular hydrogels. Examples include but are not limited to, carbomer, poloxamer, and/or hyaluronate.

In some embodiments, the polymer is carbomer (poly(acrylic acid) (PAA)). PAA is a synthetic high-molecular weight polymer of acrylic acid. The IUPAC name is poly(1-carboxyethylene). They may be homopolymers of acrylic acid, or crosslinked with an allyl ether of pentaerythritol, allyl ether of sucrose, or allyl ether of propylene.

Hyaluronate or hyaluronic acid (HA) is an anionic, nonsulfated glycosaminoglycan that is biodegradable and biocompatible.

In some embodiments, the polymer is a poloxamer. Poloxamers are biodegradable and biocompatible nonionic triblock copolymers composed of a central hydrophobic chain of polyoxypropylene (poly(propylene oxide)) flanked by two hydrophilic chains of polyoxyethylene (poly(ethylene oxide)).

Additional suitable polymers for hydrogels include, for example,chitosan, carboxymethyl chitosan, N, O-carboxymethyl chitosan, trimethyl chitosan, hydroxypropyltrimethylammonium chloride chitosan, thiolate chitosan, cellulose, methyl cellulose, ethyl cellulose, hydroxymethyl cellulose, sodium alginate, poly (ethylene glycol) (PEG), polyvinylpyrrolidone, dextran, arabic gum. poloxamers, poly(N-isopropyl acrylamide) (PNIPAAm), poly(acrylamide) (PAAm), poly-methacrylic acid (PMAA), (PAA), poly(N-isopropylacrylamide or a combination thereof.

In some embodiments, resatorvid is present in the pharmaceutical composition at a concentration of 0.01% to 10% w/v or w/w (e.g., 0.05% to 5.0%, 0.1 to 5%, or 0.5 to 5%)).

TABLE 1

| | Resatorvid Drug Concentration |
|---|---|
| Topical Platform | (w/w or w/v) |
| PenCream | 5% and lower |
| Dermabase cream | 5% and lower |
| Propylene Glycol liquid soln | 5% and lower |
| Lotion | 5% and lower |
| Carbomer Hydrogel | 2.5% and lower |
| Poloxamer Hydrogel | 2.5% and lower |
| Hyaluronate Hydrogel | 2.5% and lower |

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable for oral, rectal, nasal, topical (including transdermal, buccal and sublingual), vaginal, and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

In addition, in some embodiments, incorporation of formulations is integrated with a topical delivery device such as a transdermal skin patch for skin drug delivery or microneedle skin patch (See e.g., Akhtar et al., Biomed Tech (Berl) 2020 May 26;65(3):243-272; herein incorporated by reference in its entirety). Transdermal patches, including iontophoretic and electrophoretic devices, are further described in U.S. Pat. Nos. 6,267,983, 6,261,595, 6,256,533, 6,167,301, 6,024,975, 6,010715, 5,985,317, 5,983,134, 5,948,433 and 5,860,957; each of which is herein incorporated by reference in its entirety.

Pharmaceutical compositions for topical administration according to the present invention are optionally formulated as ointments, creams, suspensions, lotions, powders, solutions, pastes, gels, sprays, aerosols or oils. In alternative embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include ethanol, dimethylsulfoxide (DMSO), surfactants, and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprise a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 20, Tween 80, Span 80, PVA (polyvinylalcohol), glycerol, fatty acids, (e.g. oleic acid), cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus, creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate. Likewise, those for vaginal administration may be presented as pessaries, creams, gels, pastes, foams or spray formulations containing in addition to the agent, such carriers as are known in the art to be appropriate.

Formulations suitable for nasal or pulmonary administration, wherein the carrier is a solid, include coarse powders having a particle size, for example, in the range of about 20 to about 50 microns which are administered i.e., by rapid inhalation (e.g., forced) through the nasal passage from a container of the powder held close up to the nose or nano to 10 micon particles for oral inhalation. Other suitable formulations include, but are not limited to, nasal sprays, drops, or aerosols by nebulizer, a dry powder inhaler, and include aqueous or oily solutions of the agents.

Preferred unit dosage formulations are those containing a daily dose or unit, daily subdose, as herein above-recited, or an appropriate fraction thereof, of an agent. It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil or olive oil), minerals (e.g., Ca, Fe, K, etc.), vitamins, and other acceptable compositions (e.g., conjugated linoelic acid), extenders, and stabilizers, etc.

In certain embodiments, the present invention provides instructions for administering compositions described herein. In certain embodiments, the present invention provides instructions for using the compositions contained in a kit for the treatment or prevention of skin pathologies (e.g., providing dosing, route of administration, decision trees for treating physicians for correlating patient-specific characteristics with therapeutic courses of action). In certain embodiments, the present invention provides instructions for using the compositions contained in the kit to treat a variety of medical conditions associated with skin.

Various delivery systems are known and can be used to administer therapeutic agents of the present invention, e.g., encapsulation in liposomes, microparticles, microcapsules, receptor-mediated endocytosis, and the like. Methods of delivery include, but are not limited to topical, pulmonary, and intranasal routes. In specific embodiments, it may be desirable to administer the pharmaceutical compositions of the invention locally to the area in need of treatment.

It is contemplated that the agents identified can be administered to subjects or individuals having, susceptible to or at risk of developing skin damage and correlated conditions. When the agent is administered to a subject such as a mouse, a rat or a human patient, the agent can be added to a pharmaceutically acceptable carrier and systemically or topically administered to the subject.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is affected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

The present invention also includes methods involving co-administration of the agents (e.g., resatorvid) described herein with one or more additional active agents. Indeed, it is a further aspect of this invention to provide methods for enhancing existing therapies and/or pharmaceutical compositions by co-administering agents (e.g., resatorvid) described herein. In co-administration procedures, the agents may be administered concurrently or sequentially. In one embodiment, the agents (e.g., resatorvid) described herein are administered prior to the other active agent(s). The pharmaceutical formulations and modes of administration may be any of those described above. In addition, the two or more co-administered chemical agents, biological agents or radiation may each be administered using different modes or different formulations. When the agents (e.g., resatorvid) described herein are co-administered with another agent (e.g., as sensitizing agents), the effective amount may be less than when the agent is used alone.

The agent or agents to be co-administered depends on the type of condition being treated. For example, in some embodiments, the additional agent is a chemotherapeutic agent, anti-inflammatory agent, an antibiotic, pain reliever (e.g., NSAID) or other agent.

The agents described herein find use in the treatment and prevention of a variety of skin pathologies. The present disclosure is not limited to particular skin pathologies. Examples include but are not limited to, pathologies related to exposure to environmental stressors, photoaging and chronological aging, inflammatory dysregulation, microbial infection, or skin malignancies and premalignancies. In some embodiments, the environmental stressor is selected from, for example, solar radiation (e.g., ultraviolet, visible, or infrared), ionizing radiation, pollutants (e.g., dioxin, benzpyrene, particulate matter, or arsenic.), or natural or artificial allergens.

In some embodiments, the inflammatory dysregulation is, for example, erythema, urticaria, telangiectasia, edema, impaired skin barrier function, atopic dermatitis, contact dermatitis, eczematous dermatitis, allergic reactions, autoimmune conditions (e.g., lupus or psoriasis), scleroderma, or wounds (e.g., diabetic wounds, burns, radiation burns, etc.). In some embodiments, the microbial infection is a fungal, bacterial, or viral pathogen.

The present disclosure is not limited to particular skin malignancies and premalignancies. Examples include but are not limited to, melanoma, nonmelanoma skin cancer, actinic keratosis, or dysplastic nevi.

In some embodiments, the compositions described herein are administered to a subject diagnosed with a skin pathology such as those described above. In some embodiments, compositions are administered to subjects at risk of developing a skin pathology (e.g., a subject with excessive exposure to sun or environmental toxins or an individual with an underlying disease or condition).

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example 1

### Analysis of Resatorvid

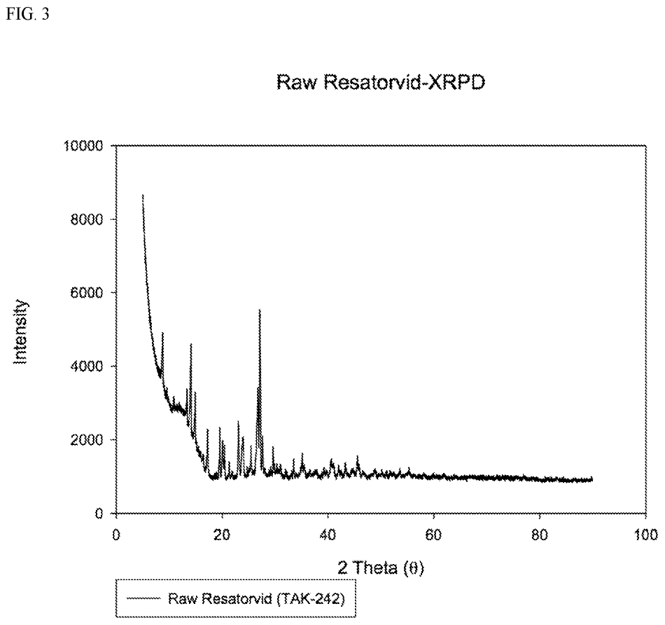

X-Ray powder diffraction (XRPD) & Karl Fischer titration (KFT) was performed. The XRPD diffractogram showed sharp peaks indicative of long-range molecular order, characteristic of a highly crystalline order (FIG. 3). To determine residual water content in resatorvid, a 0.1% w/v resatorvid solution in anhydrous MeOH was made. Results of KFT showed 0.0713±0.0156 (% w/w) residual water content (n=4), which is consistent with hydrophobicity.

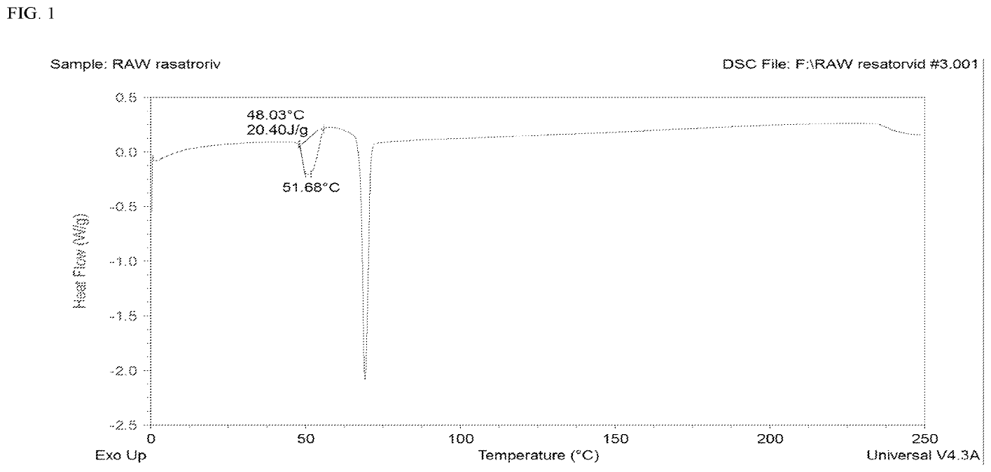

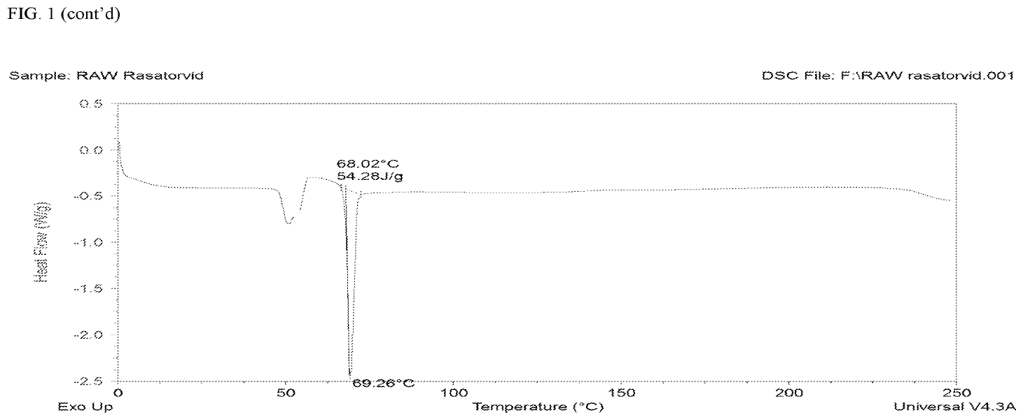

Thermal analysis by differential scanning calorimetry (DSC) was conducted. Results are shown in Table 2 and FIG. 1 and indicate two endothermic peaks, which indicates two different phase transitions. Tₚₑₐₖ of melting point of endotherm 1 is at 52.5C±1.89° C. with an enthalpy of 24.1±3.31 J/g Tₚₑₐₖ of melting point of endotherm 2 is at 69.3C±0.14° C. with an enthalpy of 57.4±2.71 J/g. Results indicate that resatorvid is a Low melting point drug compared to a typical small molecular weight hydrophobic drug.

TABLE 2
Resatorvid (TAK-242)

| | Endotherm 1 | | Endotherm 2 | |
|---|---|---|---|---|
| | (n = 3) | | (n = 3) | |
| | Tₚₑₐₖ | Enthalpy | Tₚₑₐₖ | Enthalpy |
| System | (° C.) | (J/g) | (° C.) | (J/g) |
| Raw | 52.5 ± 1.89 | 24.1 ± 3.31 | 69.3 ± 0.14 | 57.4 ± 2.71 |
| Resatorvid | | | | |

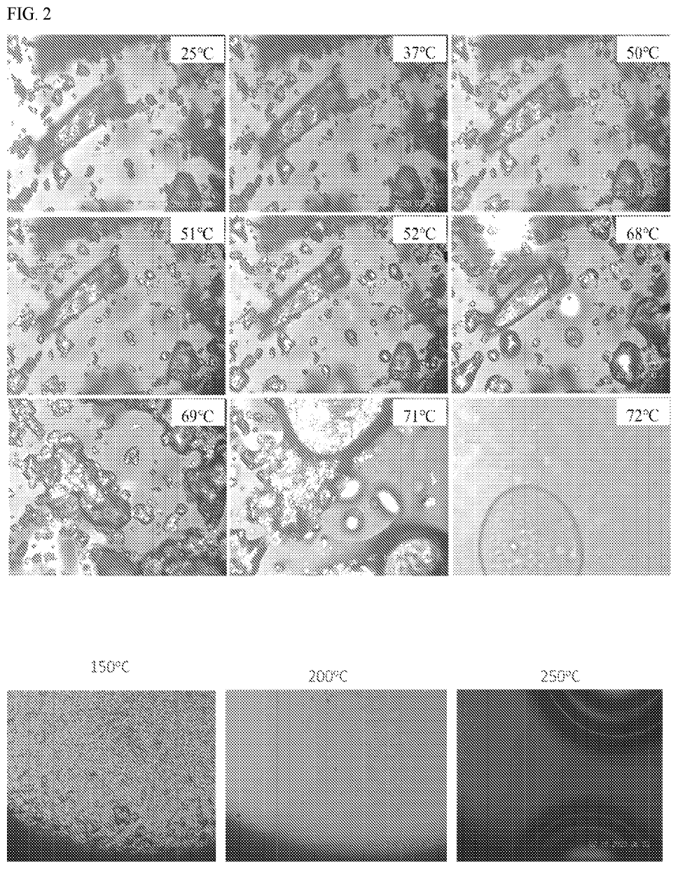

Hot-stage microscopy (HSM) under cross-polarizers was performed. Results are shown in FIG. 2. At a pretransition temp a birefringency to glassy appearance is observed (52° C.). The start of melting and main phase transition is at 69° C. Melting continues to the main phase transition (71° C.). Complete melting of drug and the end of the min phase transition is at 72° C. Drug decomposition is at 150° C.

UV/Vis spectroscopy showed that resatorvid has an absorbance in the wavelength range between 190-290 nm and no peaks at wavelengths 290 nm-700 nm (Table 3). The solvents that were used were PEG400, EtOH 140 proof, PBS pH 7.4, Acetonitrile, NS, and MeOH. PEG600 was not used as it was too thick. At a concentration of 0.1% w/v it was observed that resatorvid was soluble in PEG400, acetonitrile and EtOH 140 proof and HPLC grade MeOH. 0.01% w/v solutions were also made for some samples to show an acceptable UV-Vis spectra. Resatorvid was not soluble in NS or PBS pH 7.4. The 0.1% w/v PEG400 UV-VIS spectra shows that there was no changes in the drug after 4 days at 25° C.

TABLE 3

| Wavelength (nm) | Absorbance (n = 3) | SD |
|---|---|---|
| 280 | 1.3076 | 0.176 |
| 290 | 0.845766667 | 0.106 |
| 300 | 0.244133333 | 0.024 |
| 310 | 0.076133333 | 0.004 |
| 320 | 0.0655 | 0.003 |
| 330 | 0.060866667 | 0.003 |
| 340 | 0.052866667 | 0.002 |
| 350 | 0.0467 | 0.002 |
| 360 | 0.043133333 | 0.002 |
| 370 | 0.040966667 | 0.002 |
| 380 | 0.039633333 | 0.002 |
| 390 | 0.0384 | 0.002 |
| 400 | 0.037566667 | 0.002 |
| 450 | 0.034633333 | 0.001 |
| 500 | 0.0327 | 0.001 |
| 550 | 0.031266667 | 0.001 |
| 600 | 0.030466667 | 0.001 |
| 650 | 0.029933333 | 0.001 |
| 700 | 0.0295 | 0.001 |

HPLC analysis was performed with HPLC column: C18, 5 μm column (150 mm×2.1 mm) maintained at 25±2° C. The mobile phase was 40:60 Acetonitrile and 0.1% v/v Trifluoroacetic acid in miliq water; 500 mL total volume; the mobile phase was adjusted with 100% Acetonitrile via HPLC system to a ratio of 90:10 to give a final ratio of 50:50 (Acetonitrile to 0.1% v/v TFA in MilliQ water). The flow rate was 0.3 mL/min, the detection wavelength was 254 nm, and the injection volume was 10 μL. A 5-point calibration curve was constructed and done in triplicate (n=3). For the calibration curve a 1 mg/mL stock solution of resatorvid was made by dissolving 1 mg of drug in acetonitrile (ACN). Serial dilution as performed where the starting concentration of 1 mg/mL was diluted by half each time by using CAN.

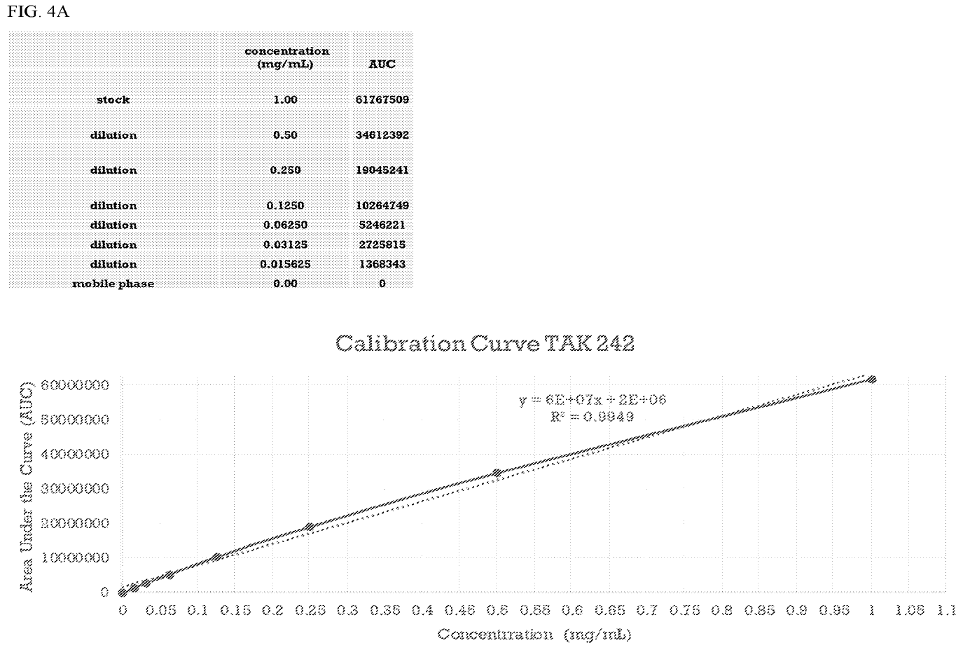

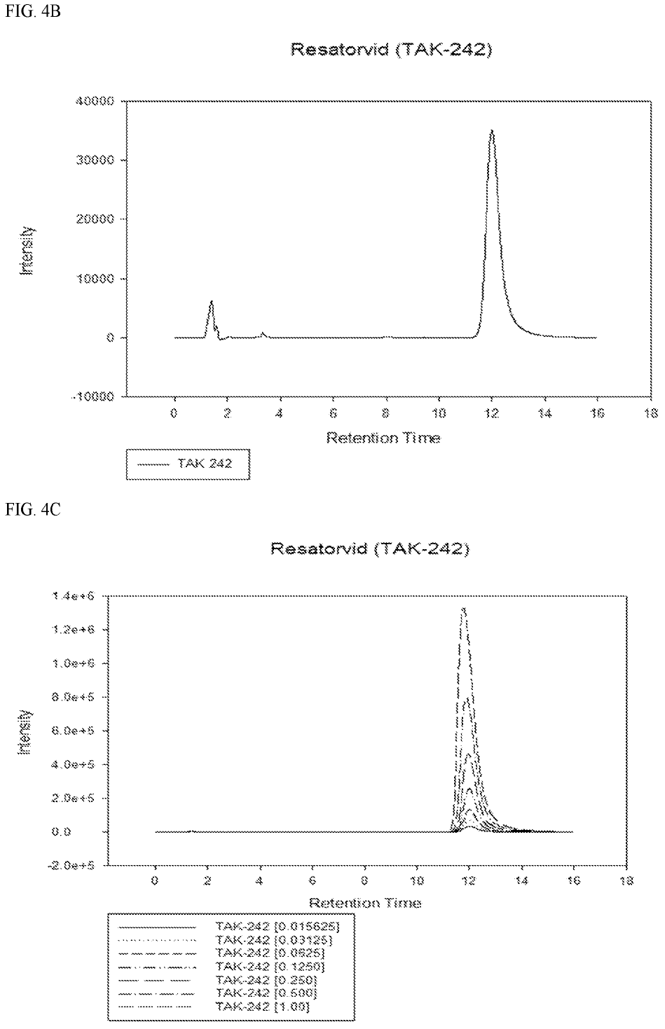

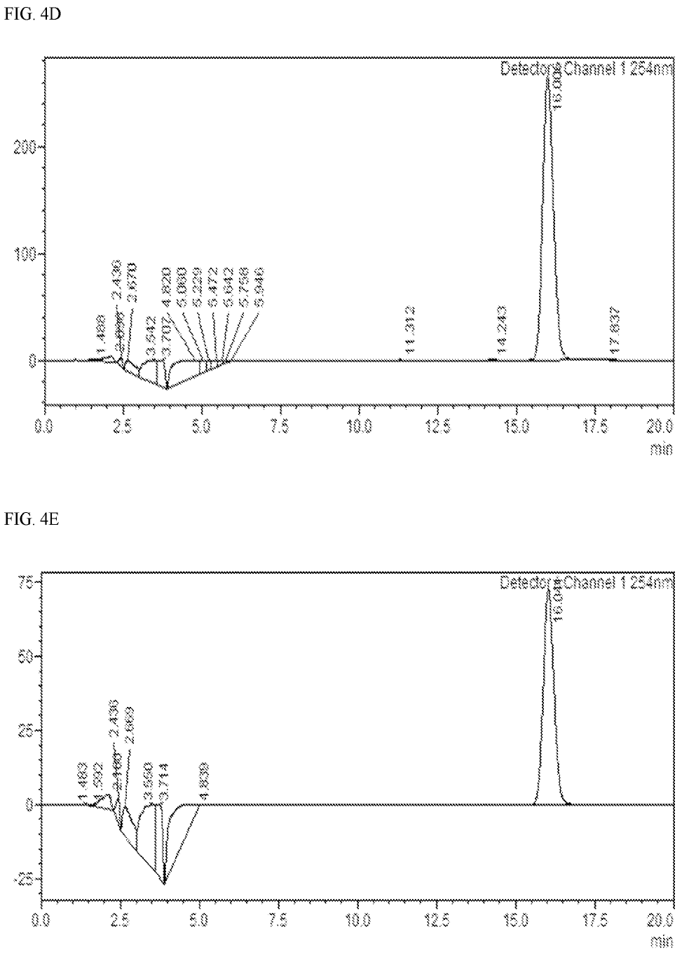

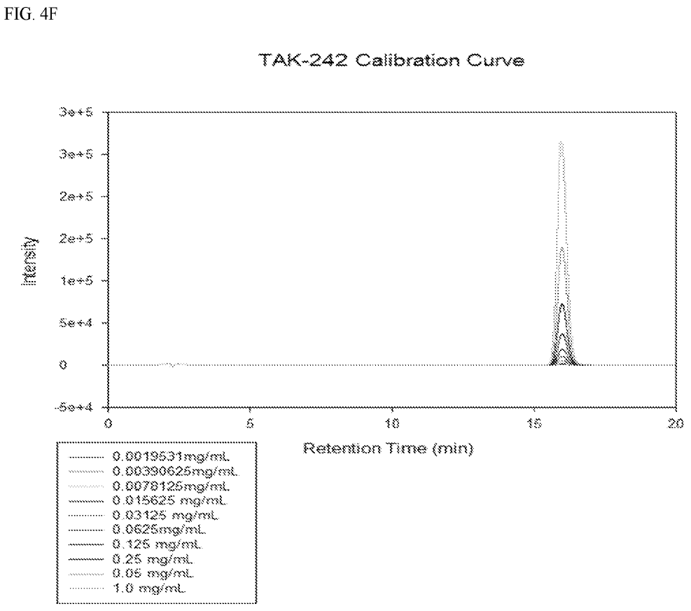

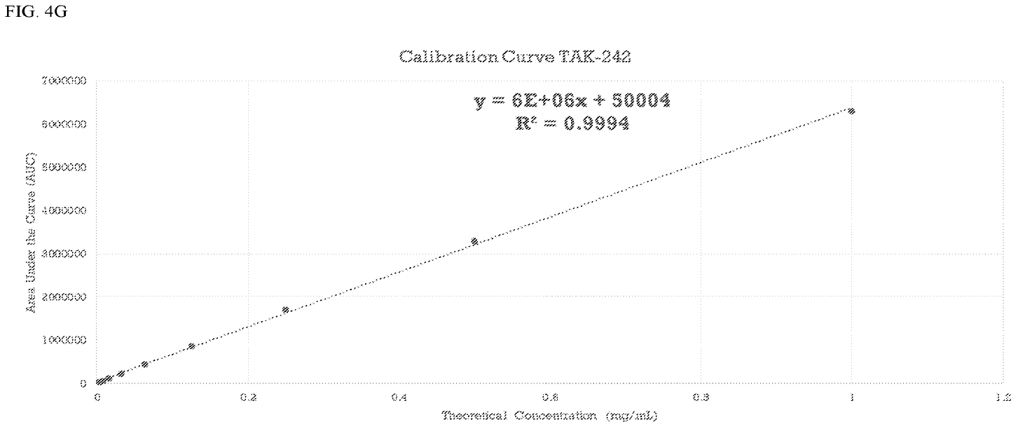

Results are shown in FIGS. 4A-D. FIG. 4A shows a calibration curve of resatorvid concentration. FIG. 4B shows 1 run of TAK 242 and FIG. 4C shows the drug at different concentrations for the calibration curve. Overall the retention time of resatorvid (TAK-242) was 11.9±0.09 min (Tables 4 and 5).

FIGS. 4D-G show further HPLC analysis of resatorvid with a column 250 mm in length (all other parameters were as described above). The retention time of resatorvid was 15.99±0.01 min with a 250 mm column length.

TABLE 4

| Point | Retention time (min) |
|---|---|
| 1 | 11.994 |
| 2 | 12.016 |
| 3 | 12.007 |
| 4 | 11.996 |
| 5 | 11.953 |
| 6 | 11.886 |
| 7 | 11.774 |
| AVE | 11.94657143 |
| SD | 0.088181361 |

TABLE 5

| Theoretical | | Calculated | |
|---|---|---|---|
| concentration | | Concentration | |
| (mg/mL) | AUC | (mg/mL) | Accuracy |
| 1.00 | 61767509 | 0.9961 | 99.6125 |
| 0.50 | 34612392 | 0.5435 | 108.7080 |
| 0.250 | 19045241 | 0.2841 | 113.6349 |
| 0.1250 | 10264749 | 0.1377 | 110.1967 |
| 0.06250 | 5246221 | 0.0541 | 86.5659 |
| 0.03125 | 2725815 | 0.0121 | 38.7101 |
| 0.015625 | 1368343 | 0.0105 | 67.3767 |
| 0.00 | 0 | 0 | 0 |

### Example 2

### Formulations

A variety of liquid, cream, and lotion formulations of resatorvid were prepared.

To determine solubility of resatorvid in a liquid formulation, a variety of solvents were tested: phosphate buffered saline, NS, ethanol, and PEG400, all with resatorvid at a concentration of 0.1% w/v and the solutions were sonicated for 1 hour at room temperature. It was observed that TAK-242 0.1% w/v failed to dissolve with PBS and NS (normal saline).

In addition, a solution of 5% w/v resatorvid in EtOH 140 proof was prepared (1 mL of ETOH and 50 mg of resatorvid). The solution was not translucent and became opaque/cloudy and there was still drug undissolved floating around. It was observed that TAK-242 at 5% only partially dissolved in EtOH but completely dissolved at 0.1% w/v TAK-242.

Solutions were made with w/v TAK242+PEG600 and 5% w/v TAK242+100% Acetone. All three solutions were sonicated at room temperature for 1 hour. The solution with acetone dissolved the drug completely but became very opaque/non-translucent showing that it was fully saturated. The solution with PEG600 dissolved the drug completely and its appearance was clear and translucent but it remained completely dissolved. As the solution began to adjust to ambient temperature it started to solidify into a white thick substance almost like lard.

Further experiments were conducted using glycerol as the solvent. 0.0504 g of TAK-242 was weighed out to make a 5.04% w/v solution in 1 mL of glycerol, USP grade. in 1 mL of glycerol, USP grade. The solution was then sonicated for 60 minutes at 37° C. If the drug did not dissolve after sonication, more glycerol was added to reach 2.5% w/v, 1.25% w/v, and 0.625% w/v and sonication was repeated. Images were taken at each to document the outcome. Results indicated that the resatorvid did not dissolve under any of the conditions tested.

The solubility of the drug as powder was tested on both Dermabase cream as well as PENcream. After compounding the powder with creams and letting them sit at room temperature for 30 minutes to 1 hour it was observed that the drug Resatorvid (TAK-242) powder was undissolved and incompatible with creams. It was a clear indication that the drug would have to be pre-dissolved in a solution before incorporating into the creams.

The miscibility of PEG 400 liquid and PEG 600 liquid in both Dermabase cream and PENcream was tested to see not only compatibility but also maintenance of cream consistency. After compounding PEG400 liquid and PEG 600 liquid into the creams and letting them sit at room temperature for 1 hour, it was observed that at 0.5 mL and 1 mL of PEG 400 Dermabase cream managed to mix well and maintain initial consistency. PENcream at a volume of 0.5 mL of PEG 400 liquid managed to maintain consistency and mixed well but failed with volumes higher than that. PEG 600 liquid was incompatible with both creams as they lost consistency and did not mix well as you could see separation of PEG 600 liquid and cream.

A 5% w/v solution of resatorvid (50mg) and PEG 400 USP (1 mL) was prepared. The solution was clear in color and translucent and there were no signs of undissolved drug.

A variety of cream/lotion formulations were prepared. One gram of Dermabase cream and 0.1 gram of resatorvid were compounded together. After 1 hour of letting the mixture sit at room temp, the end result was a grainy mixture where the drug was unable to dissolve in the cream. One gram of PENcream was compounded with 0.1 grams of resatorvid. After compounding the mixture sat for 1 hour at room temp. After 1 hour, the result was a mixture with undissolved drug. PEG 400 USP and PEG 600 USP at volumes of 0.5 mL, 1 mL and 2 mL were used with 1 gram of cream base. Results indicated that the Dermabase cream maintained its consistency with 0.5 mL and 1 mL PEG 400 USP and the PENcream base maintained its consistency with 0.5 mL PEG 400 USP and loses consistency with 1 mL added. PEG 600 USP changed the cream consistency to a lotion for each cream at added volumes 0.5 mL, 1 mL and 2 mL.

Creams were compounded as Dermabase plus resatorvid in PEG400, PEG600, and propylene glycol as 5% w/v mixture. The PEG400 USP and PEG600 USP Dermabase creams maintained their consistency over 4 days while the PG USP Dermabase cream looked more like a lotion. Formulation of PENCream plus resatorvid in PEG400 USP, PEG600 USP, and PG USP as 5% w/v TAK-242 mixture were prepared. The creams had some moisture build up in the glass flask and a bit of phase separation was visible. Using 0.5 mL of each solution, the creams lost their consistency after 5 days and become more lotion like. When the creams were initially compounded, precipitation of the drug with either solution was observed. After mixing with PENCream, and sitting for 4 days, the precipitation diminished. The Dermabase cream mixed well and there was no precipitation of the drug while compounding.

### Example 3

### Properties of Formulations

### Materials and Methods

### Materials

Resatorvid (TAK-242, molecular weight=361.82 g/mol, C₁₅H₁₇CLFNO₄) was purchased from APAC Pharmaceutical LLC (Columbia, MD, USA). Propylene glycol (USP/FCC certified) was obtained from Fisher Scientific (Fair Lawn, NJ, USA). Pluronic® F-127 powder was purchased from Sigma-Aldrich (St. Louis, Missouri, USA). Sodium hyaluronate (95%) powder was purchased from Acros Organics (Geel, Belgium). Carbomer 934P (Carbopol® 934P) resin NF, polyethylene glycol 400 (PEG-8 NF, USP certified), and polyethylene glycol 600 (PEG-12 NF, USP certified) were purchased from Spectrum Chemical MFG Corp. (Gardena, CA, USA). PENcream™ (versatile topical cream for Rx compounding) was purchased from Humco (Texarkana, TX, USA). DermaBASE™ Cream (oil in water emulsion base) was purchased from Perrigo (Minneapolis, Minnesota, USA). Hydranal®-Coulomat AD and resazurin sodium salt were from Sigma-Aldrich (St. Louis, Missouri, USA).

Human keratinocyte cell lines (HaCaT, AddexBio® T0020001) were purchased from AddexBio (San Diego, CA, USA). Dulbecco's modified Eagle's medium (DMEM), Optimized 1× was obtained from AddexBio (San Diego, CA, USA). Fetal bovine serum (FBS), Pen-Strep, and fungizone were obtained from Gibco® by Life Technologies (Thermo Fisher Inc, Waltham, MA, USA). Normal human epidermal keratinocytes (NHEKO) (unique primary adult cell lines) and their growth medium (NHEK-GM®) were both purchased from MatTek Life Sciences (Ashland, MA, USA). EpiDerm™ (a 3D tissue model consisting of normal, human derived epidermal keratinocytes) and its EpiDerm™ growth media were both purchased from MatTek Life Sciences (Ashland, MA, USA). The Strat-M® membrane (Transdermal Diffusion Test Model, 47 mm) was purchased from Millipore Sigma (Danvers, MA, USA).

### Pre-Formulation of Resatorvid: Solubility and Compatibility

Seven platforms were analyzed for compatibility with TAK-242. The platforms were DermaBASE cream (cream and lotion), PENcream (cream), PEG-400 solution, PG solution, 1% w/v Carbomer 934P resin gel at pH˜7, 2% w/v Sodium hyaluronate gel/serum and 20% w/v Pluronic F127 gel. Solubility of TAK-242 was determined at concentrations ranging from 0.1-5% w/v using PBS (pH=7.41), 0.9% normal saline (NS), HPLC grade ethanol, PEG-400 and PG as solvents. The following step was to test if TAK-242 could be directly incorporated into the platforms mentioned above via non-sterile compounding to determine if the drug would need to be pre-dissolved before incorporation. Determination of how much volume of PEG-400 or PG could be incorporated into the creams and gels without losing consistency while maintaining homogeneity was examined; 1 g of base (cream or gel) was weighed out and was compounded with PEG-400 or PG (500 μL-1000 μL). Drug was then dissolved in appropriate determined volume of compatible solvent and sonicated for 60 minutes, then it was prepared via non-sterile compounding technique and formulations were examined for physical appearance (consistency, homogeneity, visible precipitation, phase separation, compatibility). The only formulation that was different was the Pluronic F127 as everything was mixed in as a liquid at 4° C. and the solution would then solidify at room temperature (˜37° C.) to create the gel which was then mixed thoroughly until homogenous.

### Preparation of Topical Formulations by Non-Sterile Compounding

A total of 8 formulations of resatorvid (TAK-242) were prepared via non-sterile compounding technique. The formulations were chosen based on drug compatibility and solubility in the base materials PEG-400 and PG after a series of test trials. TAK-242 was dissolved in 20 mL scintillation vials after water-bath sonication and a glass slab (12×12, 1.5″ thick) was used for non-sterile compounding.

DermaBASE cream formulation was made at concentrations of 1.25% w/w, 2.5% w/w and 5% w/w. TAK-242 was dissolved in PEG-600 using a Branson 2800 sonicator. Once the drug was fully dissolved the formulation was made via non-sterile compounding which is technique used to create a medication in a clean environment but does not require the environment to be completely free of microorganisms. PENcream formulation and DermaBASE lotion were prepared with the same method. The solvent for TAK-242 in DermaBASE lotion was propylene glycol (PG).

Carbomer gel formulation was made at concentrations of 1.25% w/w and 2.5% w/w. The gel base was made at a concentration of 1% w/v using carbomer 934P and milliq-water. The pH was adjusted to 7-7.4 with triethanolamine. TAK-242 was dissolved in PEG-400 using a Branson 2800 sonicator. Once the drug was fully dissolved the formulation was compounded via non-sterile compounding technique. Hyaluronic acid gel/serum formulation was made at concentrations of 1.25% w/w and 2.5% w/v. The gel/serum base was made at a concentration of 2% w/v using sodium hyaluronate (95%) and milliq-water. TAK-242 was dissolved in PEG-400 using a Branson 2800 sonicator. Once the drug was dissolved the formulation was compounded using non-sterile technique. Polaxamer gel formulation was made at a concentration of 1.25% w/v and 2.5% w/v. The gel base was made using Pluronic (F127) at a concentration of 20% w/v in milliq-water and with respect to the desired TAK-242 concentration. TAK-242 was dissolved in PEG-400 using a Branson 2800 sonicator. This formulation was not compounded but mixed with the liquid pluronic, the concentration of the gel base was kept at 20% w/v.

### Scanning Electron Microscopy (SEM) and Energy-Dispersive X-Ray (EDX) Spectroscopy

Particle size and surface morphology of raw TAK-242 were obtained using scanning electron microscopy (SEM) (FEI, Brno, Czech Republic). Samples were sprinkled onto the double-sided adhesive carbon tabs (TedPatella, Inc. Redding, CA, USA) which were adhered to aluminum stubs and coated with a 7 nm thin film of gold palladium alloy using a Anatech Hummer 6.2 (Union City, CA, USA) sputtering system at 20 μA for 90 s under an argon plasma. The electron beam with an accelerating voltage of 30 kV was used at a working distance of approximately 9-12 mm. Several magnifications were used to get the best resolution.

EDX was performed using ThermoNoran systems Six (Thermo Scientific, Waltham, MA, USA) at accumulation voltage of 30,000 eV, the spot size was increased until a dead time of 20-30 was obtained.

### Particle Sizing and Size Distribution by SEM Image Analysis

Using SEM micrographs, the mean size, standard deviation, and size range of the particles were determined digitally using SigmaScan™ Pro 5.0.0 (Systat, Inc., San Jose, CA, USA). Representative micrographs of raw TAK-242 at 400-5000× magnification were analyzed by measuring the diameter of at least 60 particles per sampling.

### X-Ray Powder Diffraction (XRPD)

The powder crystallinity of raw TAK-242 was determined by X-ray powder diffraction (XRPD). XRPD patterns of raw TAK-242 were collected at room temperature with a PANalytical X'pert diffractometer (PANalytical Inc., Westborough, MA, USA) equipped with a programmable incident beam slit and an X'celerator detector. The x-ray radiation used was Ni-filtered Cu Kα (45 kV, 40 Ma, and λ=1.5444 Å). Measurements were taken between 5° and 89.9° (2θ) with a scan rate of 2°/min. The powder samples were loaded on zero background silicon sample holder.

Differential Scanning calorimetry (DSC)

A TA Q1000 differential scanning calorimeter (DSC) (TA Instruments, New Castle, DE, USA) equipped with T-Zero® technology, RSC90 automated cooling, and an auto sampler was used to perform thermal analysis and phase transition measurements for raw TAK-242. The instrument was previously calibrated with indium. Approximately 1-3 mg of raw TAK-242 was weighed and placed into anodized aluminum hermetic DSC pans. The T-Zero® DSC pans were hermetically sealed with T-Zero® hermetic press (TA Instruments, New Castle, DE, USA). An empty hermetically sealed aluminum pan was used as a reference. UHP nitrogen gas used as the purging gas at a rate of 40 mL/min. The samples were heated from 0° C. to 250° C. at a scanning rate of 5° C./min. All measurement were carried out in triplicate.

### Hot-Stage Microscopy (HSM)

Hot-stage microscopy (HSM) was completed using a Leica DMLP cross-polarized microscope (Wetzlar, Germany) equipped with a Mettler FP 80 central processor heating unit and Mettler FP82 hot stage (Columbus, OH, USA). Raw TAK-242 was sprinkled onto a glass slide and heated from 25° C. to 250° C. at a heating rate of 5° C./min. The images were digitally captured using a Nikon Coolpix 8800 digital camera (Nikon, Tokyo, Japan) under 10× optical objective and 10× digital zoom.

### Karl Fisher Titration (KFT)

Karl fisher titration (KFT) was used to analyze the residual water content of raw TAK-242 powder. It was chemically quantified by coulometric KFT using a TitroLine 750 trace titrator (SI Analytics, Weilheim, Germany). A solution of 0.1% w/v of TAK-242 in anhydrous methanol was made and then 1 mL of solution was added to the titration cell line containing Hydranal® Coulomat AD reagent.

### Raman Spectroscopy

Raman spectra of raw TAK242 were obtained at 785 nm laser excitation using Renishaw InVia Reflex (Gloucestershire, United Kingdom) at the surface using a 20× magnification objective on a Leica DM2700 optical microscope (Wetzlar, Germany) and equipped with a Renishaw inVia Raman system (Gloucestershire, United Kingdom). This system had a 2400 l/mm grating, with a slit width of 65 μm and a thermoelectrically cooled Master Renishaw CCD detector. The laser power was adjusted to achieve 5000 counts per second for the 520 cm−1 line of the internal Si reference. Raman spectra were acquired with 1% of laser power and 10 seconds of exposure for raw TAK-242 powder.

### Attenuated Total Reflectance (ATR)-Fourier-Transform Infrared (FTIR) Spectroscopy

Attenuated total reflectance Fourier transform infrared (ATR-FTIR) spectra of raw TAK242 were recorded using a Nicolet Avatar 360 FTIR spectrometer (Varian, Inc., CA, USA) equipped with a DTGS detector and a Harrick MNP-Pro (Pleasantville, New York, USA) attenuated reflectance (ATR) accessory. Each spectrum was collected at 32 scans at a spectral resolution of 2 cm−1 over the wavenumber range of 4000-400 cm−1. A background spectrum was carried out under the same experimental conditions. Spectral data was acquired with EZ-OMNIC software.

### Ultraviolet (UV)/Visible (Vis) Spectroscopy

A Molecular Devices® SpectraMax® M3 Multi-Mode Microplate Reader (Sunnyvale, CA, USA) was used to perform UV/Vis spectroscopy. The wavelength range was 190 through 290 nm. A 0.1% w/v solution of TAK-242 in HPLC grade methanol, PEG-400, HPLC grade ethanol, HPLC grade acetonitrile, PBS (pH=7.4) and 0.85% w/v normal saline were made and analyzed under UV visible spectroscopy on a 96-wellplate black clear bottom.

### High Performance Liquid Chromatography (HPLC) Analysis

The HPLC consisted of a Shimadzu LC-2010A HT liquid chromatograph (Torrance, CA, USA) coupled with a UV-Vis dual wavelength detector. The analysis was performed by a reverse phase HPLC assay, using a Luna C18, 5 μm column (250 mm×2 mm) (Phenomenex, Torrance, CA, USA), maintained at 25° C.±2° C. Ultraviolet detection was done at 254 nm. Mobile phase conditions were 50:50 (v/v) ACN: Trifluoroacetic acid (0.1% v/v) at a flow rate of 0.3 mL/min. The injection volume was 10 μL. The retention time for TAK-242 was ˜15.9 minutes. Quantification was determined using peak area and calculated from a five-point standard curve (0.0625 mg/mL to 1 mg/mL, R2=0.9971). Standards were prepared by volumetric dilution in acetonitrile and stored at 4° C., protected from light (19). In Vitro Oil-Water Partitioning Coefficient (Log-P) Analysis of Resaotrvid (TAK-242)

TAK242 raw powder was weighed out (0.003 g) and placed in 2 dram ambered colored glass vials containing equal volumes (1.5 mL) of 1-octanol and PBS (1×, pH=7.4). Various solutions were made, and pH was adjusted and maintained at 6.5, 7.1, 8.8, adjustments were made using 0.1 M HCl solution and 0.1 M NaOH solution. The temperatures used in the analysis were 37° C. and 35° C. The vials were rotated using a Barnstead/Thermolyne Labquake shaker (Dubuque, IA, USA) for 24 hours and then left undisturbed in a vertical position for an additional 24 hours for phase separation between the aqueous and non-aqueous layers. A sample of each layer was carefully withdrawn and then analyzed by a validated HPLC method as describe in the HPLC analysis section under methods.

In Vitro Permeation of Resatorvid (TAK-242) through Strat-M® Transdermal Diffusion Membrane

The permeation of TAK242 from the prepared formulations through Strat-M® transdermal diffusion membrane. The receptor compartment was 5 mL and the effective diffusion area was 0.64cm². The TAK-242 formulations were prepared as outlined in the preparation of topical formulations by non-sterile compounding section under the methods section. A mixture of HPLC grade ethanol (10% v/v) and phosphate buffered saline (PBS) at pH 7.4 (1×) was used as the receptor medium, which was maintained at 37° C.±0.05° C. using a Precision reciprocal shaking bath model 25 (Thermo Fisher Scientific, Fair Lawn, NJ, USA) and oscillated at a constant speed of 30 oscillations per minute during the experiment. At predetermined time intervals, 200 μL of the receptor medium were removed and replaced with an equal volume of fresh media. The cumulative amounts of TAK-242 that permeated through the Strat-M® membrane were plotted as a function of time and permeation parameters were determined using a validated HPLC method as described in the HPLC analysis section. Flux at steady state (JT) was estimated as the slope of the linear regression analysis of the linear portion of the permeation curveLag time (tL) was defined as the time intercept of the steady-state region of the permeation curve (i.e., x-intercept). The amount of TAK-242 left on the Strat-M® membrane was extracted and quantified using a validated HPLC method as described in the HPLC analysis section.

### In Vitro Cell Dose-Response Assay in 2-D Cell Culture

The effects of raw TAK-242 powder on the viability of human representative human keratinocyte cell lines exposed to different concentrations were tested. HaCaT (a human transformed keratinocyte from histologically normal skin cell line) and NHEK (a primary normal epidermal keratinocyte cell line) were used as models of the human epidermis, which express keratinocytes in the stratum basale and then move up the final barrier layer of the skin, which is the stratum corneum. The HaCaT cells were grown in Dulbecco's modified Eagle's medium (DMEM), Optimized 1×, 10% (v/v) fetal bovine serum (FBS), and Pen-Strep (100 units/mL penicillin, 100 μg/mL) in a humidified incubator at 37° C. and 5% CO₂. NHEK cells were grown NHEK- growth medium provided by MatTek (Ashland, MA, USA) in a humidified incubator at 37° C. and 5% CO₂.

After confluence, HaCaT and NHEK cells were seeded in 96-black well plate at a concentration of 5,000 cells in 100 μL of media per well. After 48 hours, cells were then incubated for 48 h to allow attachment to the surface of the plates. Cells were then exposed to different concentrations of TAK-242. The drug solutions were prepared by dissolving raw TAK-242 powder in 5% v/v DMSO and DMEM media to make a solution at 1×. A volume of 100 μL of drug solution was added to each well. After 72 h of exposure and incubation at 37° C. and 5% CO₂, 20 μL of 20 μM resazurin sodium salt were added to each well and incubated for 4 h. Fluorescence intensity of resofurin was detected at 544 nm (excitation) and 590 nm (emission) using the Molecular Devices® SpectraMax® M3 Multi-Mode Microplate Reader (Sunnyvale, CA, USA). The relative viability of the cells was calculated as followed by equation 1:

$$Relative Viability \% = \frac{Sample flourescence intensity}{Control flourescece intensity} \times 100 \% \qquad ( Equation 1 )$$

In Vitro Permeation of Resatorvid (TAK-242) through 3-D Normal Human Derived Epidermal Keratinocytes (EpiDerm™)

After receiving EpiDerm™ 3-D normal human derived epidermal keratinocytes and following Mat-Tek's protocol, tissue culture inserts were transferred to a 6-well cell culture plate that would serve as the receptor compartment and was pre-filled with 1 mL of Dulbecco's phosphate buffered saline (PBS, 1×) without CaCl₂ and MgCl₂ (Mat-Tek, Ashland, MA, USA) media and placed inside a precision reciprocal shaking bath model 25 (Thermo Fisher Scientific, Fair Lawn, NJ, USA) with oscillation speed of 30 oscillations per minute and at 37° C.±0.05° C. This set up is to mimic that of a Franz cell diffusion cell where there is a donor/receiver interface. The effective diffusion area was 0.256 cm². The 1.25 TAK-242 formulations were prepared as outlined in the preparation of topical formulations by non-sterile compounding section under the methods section. At predetermined time intervals, 200 μL of the receptor medium were removed and replaced with an equal volume of fresh media. The cumulative amounts of TAK-242 that permeated through the EpiDerm™ were plotted as a function of time and permeation parameters were determined using a validated HPLC method as described in the HPLC analysis section. Flux at steady state (JT) was estimated as the slope of the linear regression analysis of the linear portion of the permeation curveLag time (tL) was defined as the time intercept of the steady-state region of the permeation curve (i.e., x-intercept). The amount of TAK-242 left on the EpiDerm™ was extracted and quantified using a validated HPLC method as described in the HPLC analysis section.

### Statistical Analysis

The data are presented as the mean±one standard deviation, derived from three independent experiments. The statistical analysis among groups was performed using a one-way analysis of variance (ANOVA). A value of p<0.05 was considered as significant.

### RESULTS

### Scanning Electron Microscopy (SEM) and Energy-Dispersive X-Ray (EDX) Spectroscopy

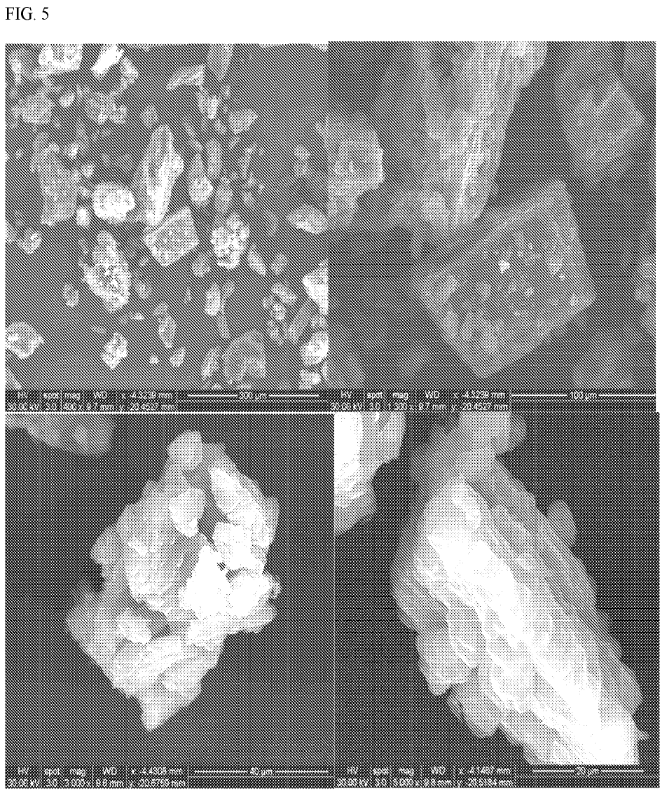

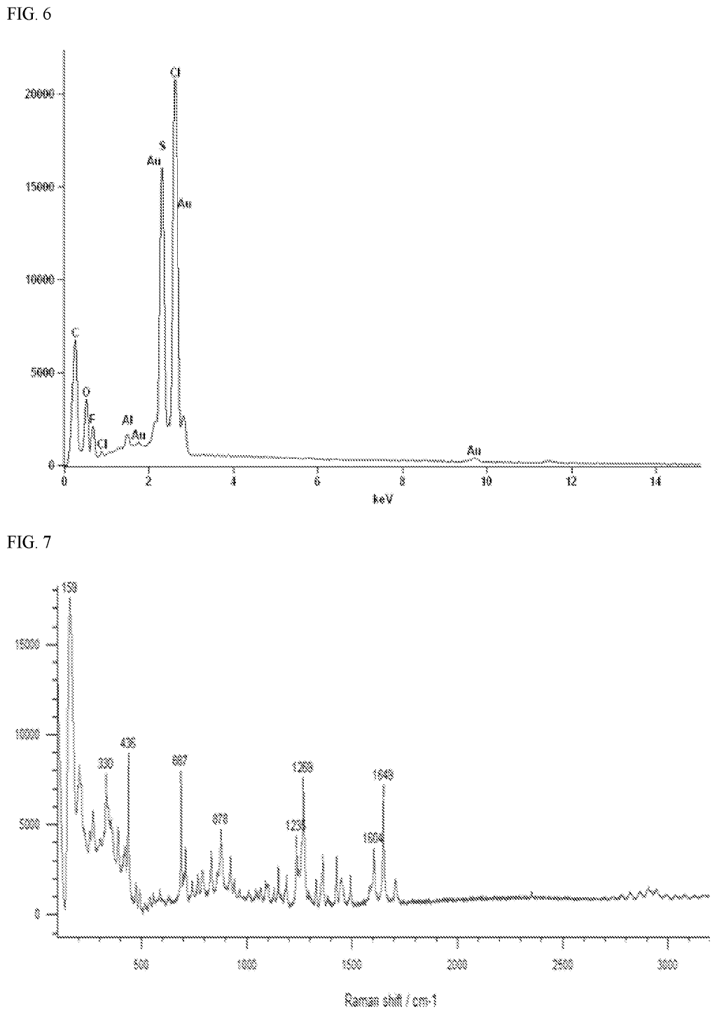

The SEM micrographs showed variable change in particle shape and size in raw TAK-242 powder as shown in FIG. 5. TAK-242 had characteristics of a crystal with a shape ranging from square to needle-like. The geometric (linear) size was micrometers for raw TAK-242 powder with an average of 5.84 μm (range: 0.024 μm to 349 μm). The Energy-Dispersive X-ray (EDX) spectra of TAK-242 is shown in FIG. 6. For chemical identification of TAK-242, the characteristic Kα line (peaks) of chlorine (Cl) is seen at 2.6 keV, the Kα line of fluorine (F) is seen at 0.68 keV and the Kα line of sulfur (S) is seen at 2.3 keV. The Kα line of carbon (C) is seen at 0.3 keV and the Kα of oxygen (O) is seen at 0.5 keV. The peaks corresponding to S, Cl, F are representative of TAK-242.

### X-Ray Powder Diffraction (XRPD)

The XRPD diffraction patterns of raw TAK-242 powder presented sharp and intense peaks, characteristic of the crystalline as illustrated on FIG. 3. All raw TAK-242 had the same diffraction pattern and sharp, intense, and notable peaks at 2θ angles 13.66°, 19.65°, 25.2°, 27.2°, and 30.78°.

Differential Scanning calorimetry (DSC)

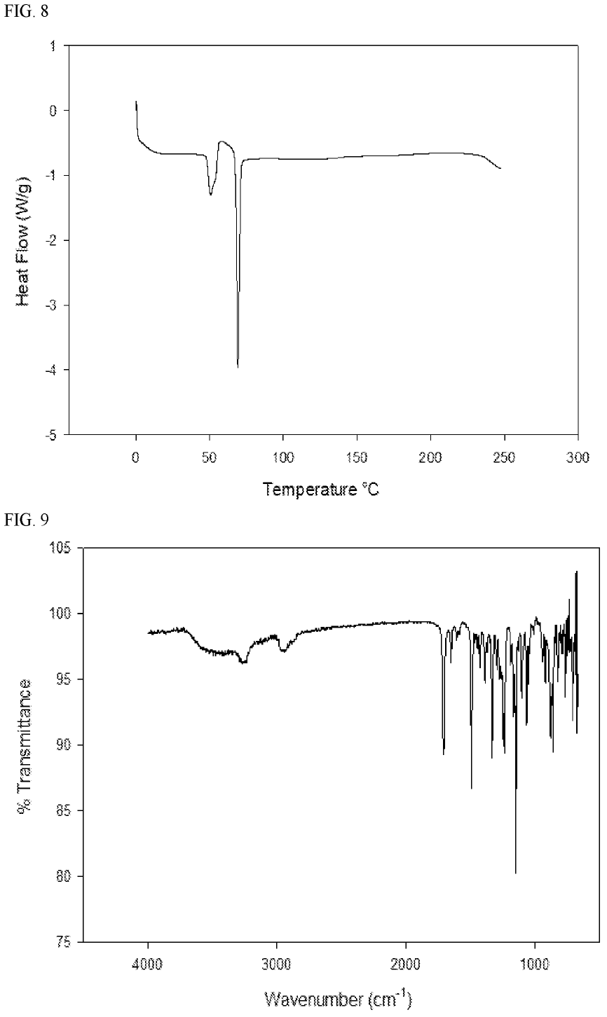

As shown in FIG. 8, raw TAK-242 exhibited two endothermic transitions at ˜53° C. and 69° C. in the DSC thermograms. The transition at 53° C. corresponds to a crystalline/glass phase transition from a solid to glass and the transition at 69° C. corresponds to the melting of the drug from a glass state to a liquid state. TAK-242 exhibited two endotherms between 53° C.-69° C. suggesting a phase transition from ordered to disordered. The enthalpy and temperature values are summarized in Table 6. The predicted melting of TAK-242 is ˜69° C.

### Hot-Stage Microscopy (HSM)

As visualized by HSM, glass transition of raw TAK-242 was observed at 50-52° C. as shown in FIG. 2, which was consistent with DSC thermogram. The raw powder possessed birefringence while melting and the melting completed, as mentioned previously at ˜71° C. which is confirmed by droplet formation and disappearance of birefringence. The residual water content of raw TAK-242 was quantified by Karl Fisher coulometic titration.

### Karl Fisher Titration (KFT)

As seen in Table 7, raw TAK-242 had an average water content of 0.0713% w/v±0.0156.

### Raman Spectroscopy

Characteristic Raman bands appeared in different wavelengths of the spectrum (FIG. 7). The most representative for TAK-242 were at 878 cm−1 (C—Cl antisymmetric stretch), at 1649 cm⁻¹ (Ar—NH bend), at 1235 cm⁻¹ (COC stretching), at 1269 cm⁻¹ (Aromatic C—H bend). The assignment of peaks in the IR spectrum is difficult and may be inaccurate due to overlapping in bands. Furthermore, some distinctive characteristic peaks of TAK-242 were identified.

### Ultraviolet (UV)-Visible Spectroscopy

ATR-FTIR spectrum are shown in FIG. 9. At approximately 1492 cm−1 (SO2 antisymmetric stretching) characteristics of sulfonamide group. The peaks at range of 1244 cm−1 to 1149 cm−1 a C—F stretch is observed. A slight peak at 1706 cm−1 (C═O stretch) is indicative of α-β unsaturated ketone. The spectral pattern seen at the fingerprint region (<2000 cm−1) was consistently observed in raw TAK-242.

### In Vitro Oil-Water Partitioning Coefficient (Log-P) Analysis of Resaotrvid (TAK-242)

The predicted pKa of TAK-242 was 6.5887 using Molecular Operating Environment (MOE®) software (Montreal, Canada) and the experimentally determined pKa reported was 8.0-8.1. The predicted log-P (cLogP) of TAK-242 was 2.53 using ChemDraw™ Ver. 16.0 (Cambridge Soft, Cambridge, MA, USA) and 3.04 using Swiss ADME (Swiss Institute of Bioinformatics, Switzerland). The partition coefficient of TAK-242 was determined using 3 different pKa's (6.5, 7.1, 8.8) based on no primary literature determining the nature of it. As shown in Table 8 the partition coefficient (Log-P) of TAK-242 at 35° C. is within range of 0.94-1.68 and at 37° C. is within range of 0.88-1.58.

### Ultraviolet (UV)/Visible (Vis) Spectroscopy

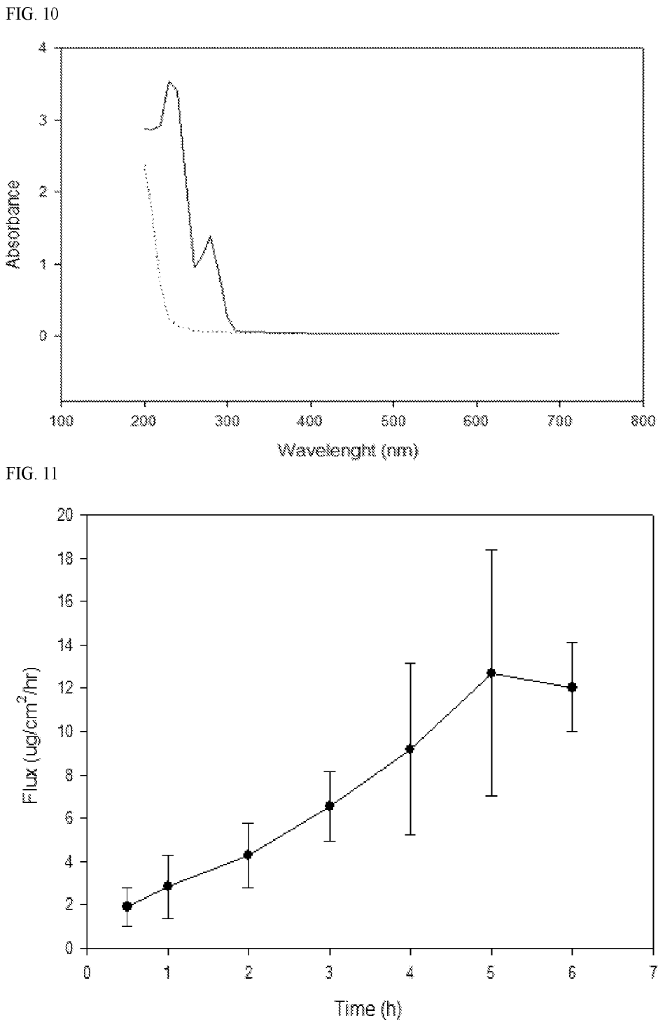

Ultraviolet-visible spectroscopy (UV-vis) showed there was significant absorption below wavelength of 250-250 nm (UVA) and a minor lambda max in the UVB region at 280 nm with a decrease in absorption until lambda of 300 nm as shown in FIG. 10.

### High Performance Liquid Chromatography (HPLC) Analysis

High performance liquid chromatography (HPLC) analysis showed TAK-242 had an average retention time of 15.9±0.01 minutes as shown in FIG. 4B.

In Vitro Permeation of Resatorvid (TAK-242) through Strat-M® Transdermal Diffusion Membrane

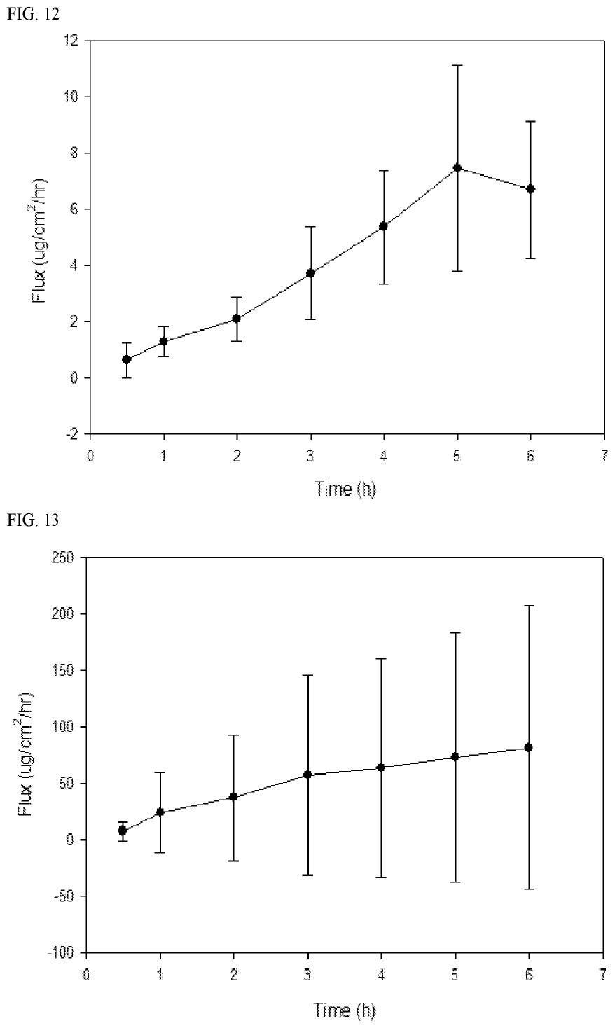

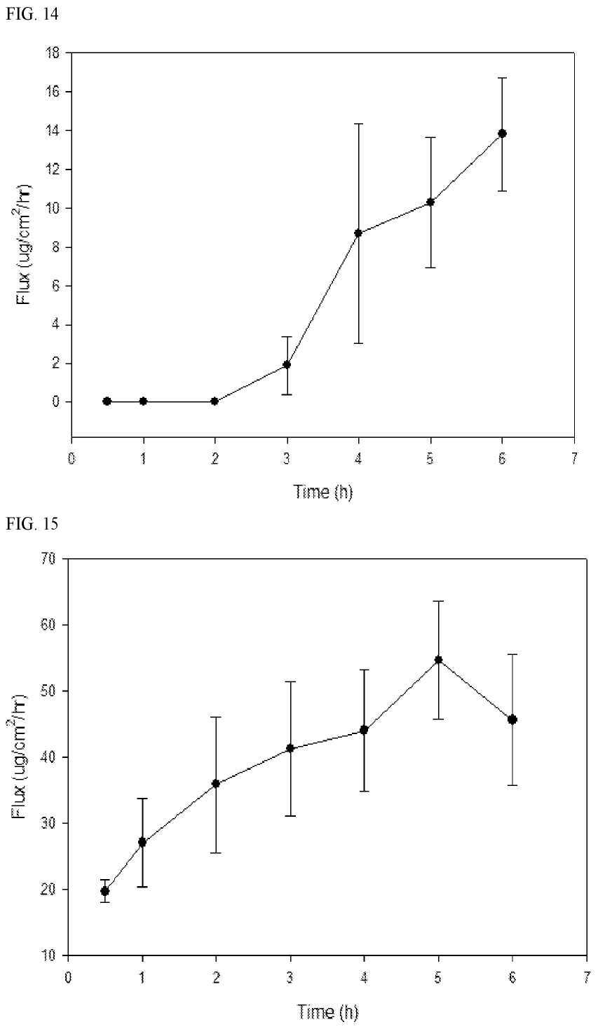

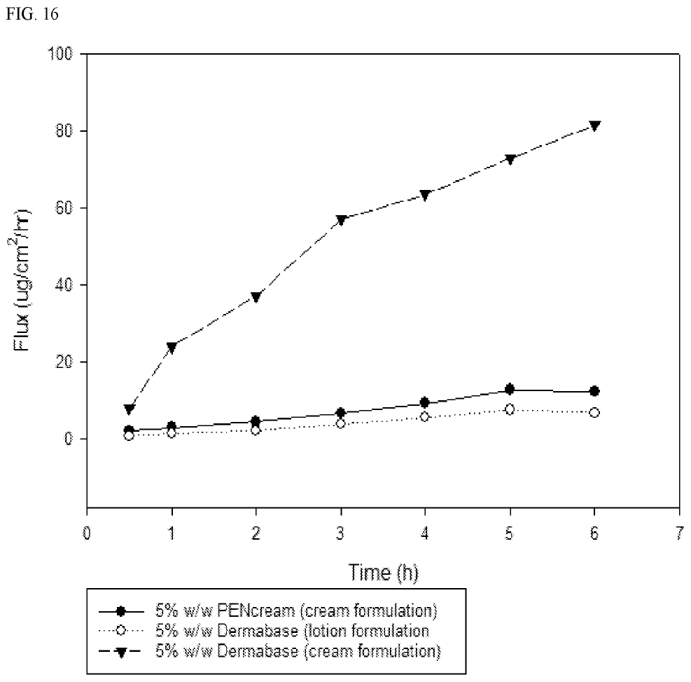

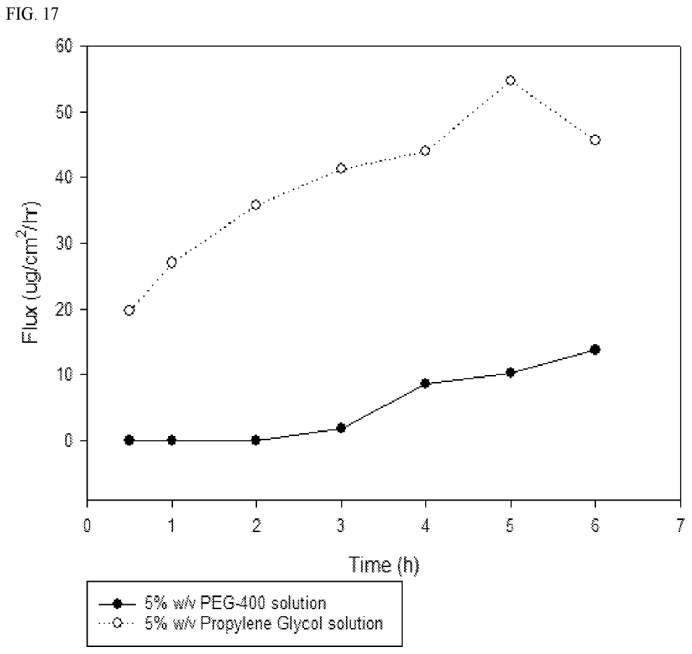

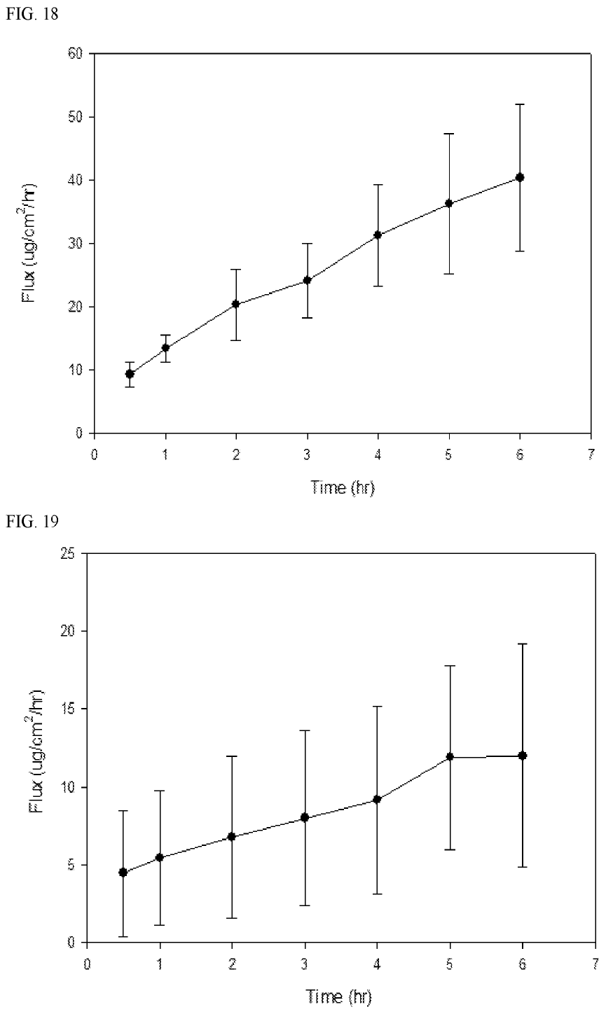

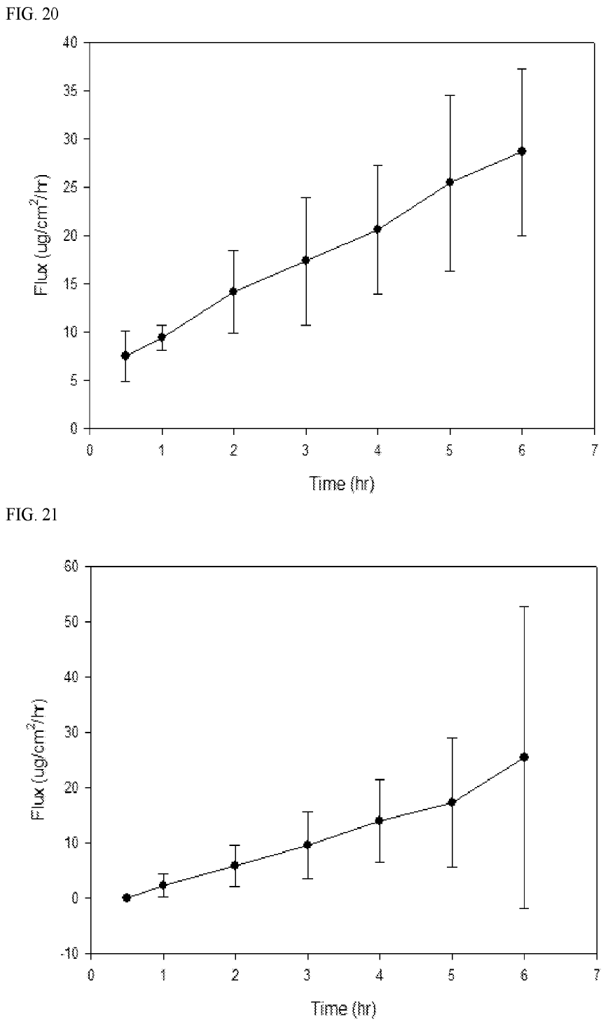

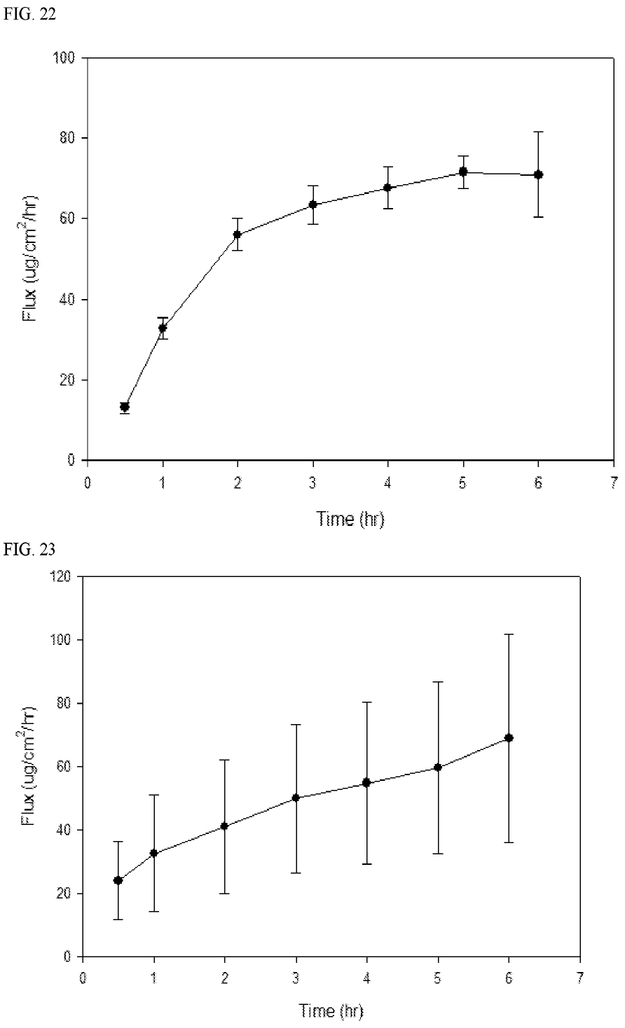

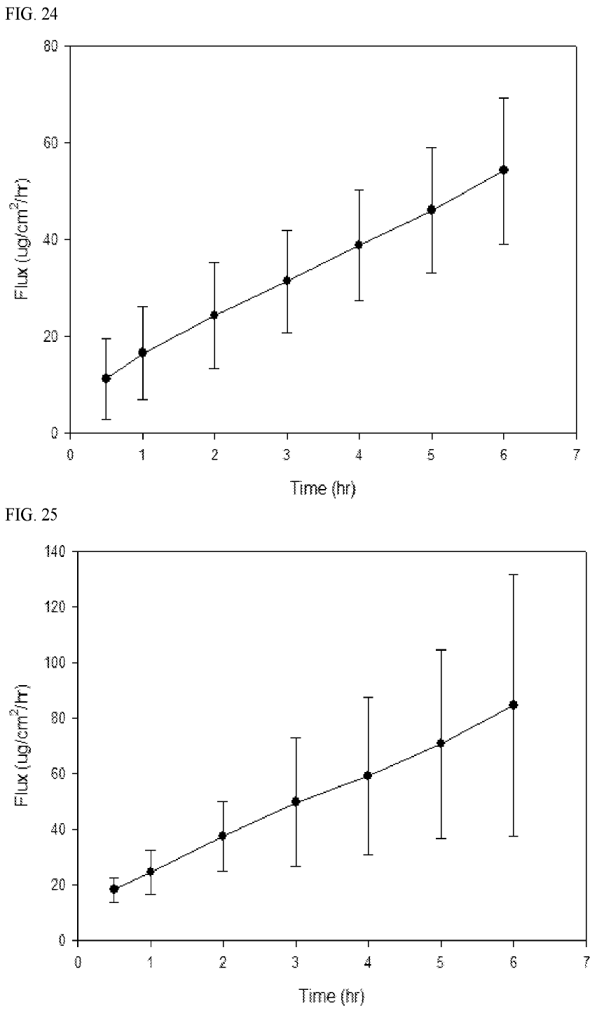

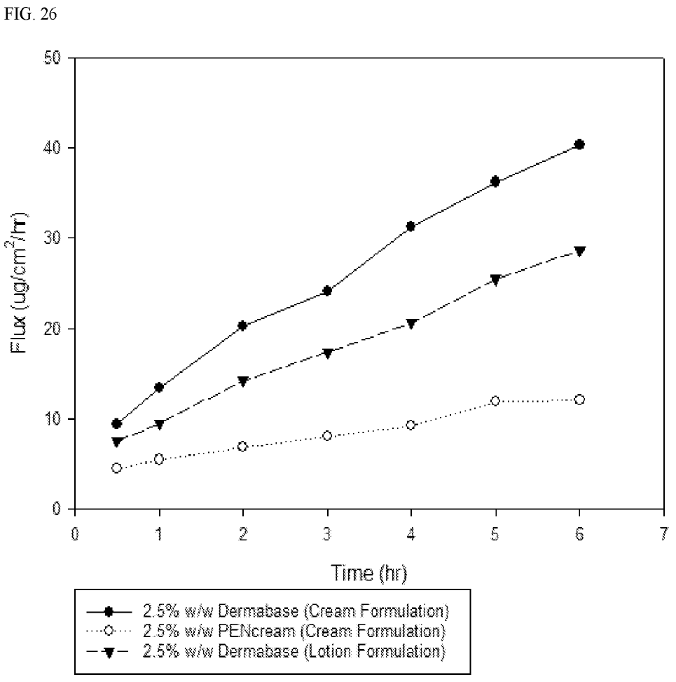

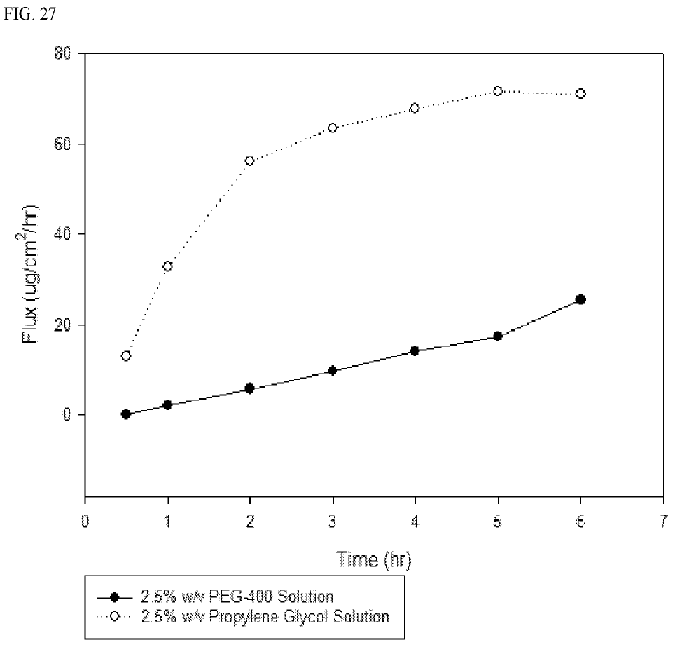

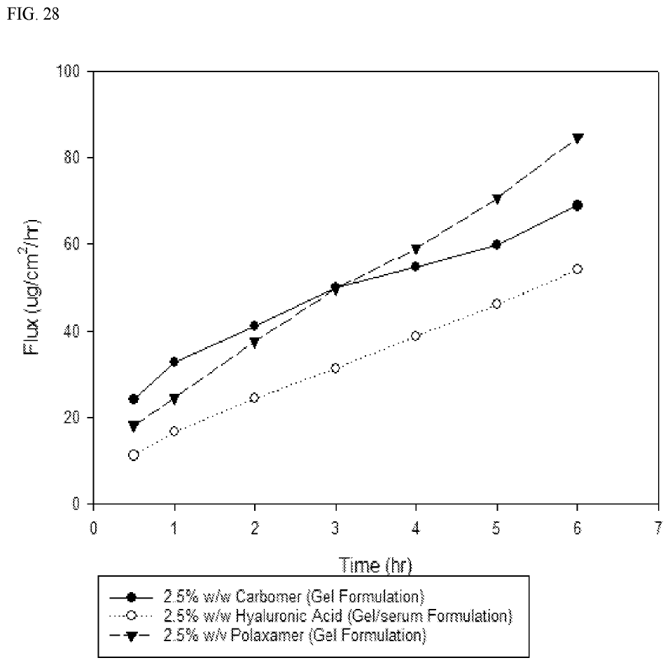

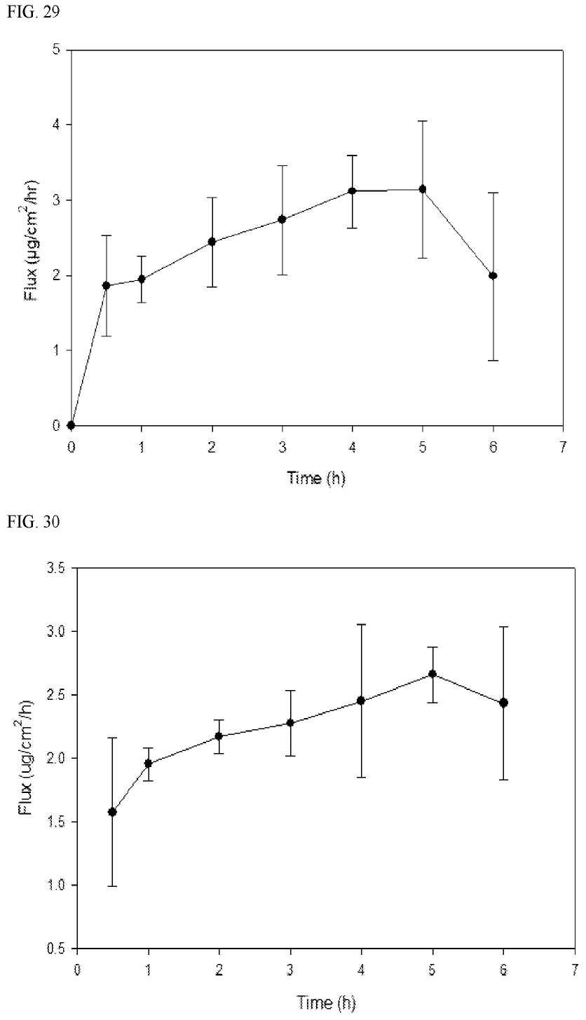

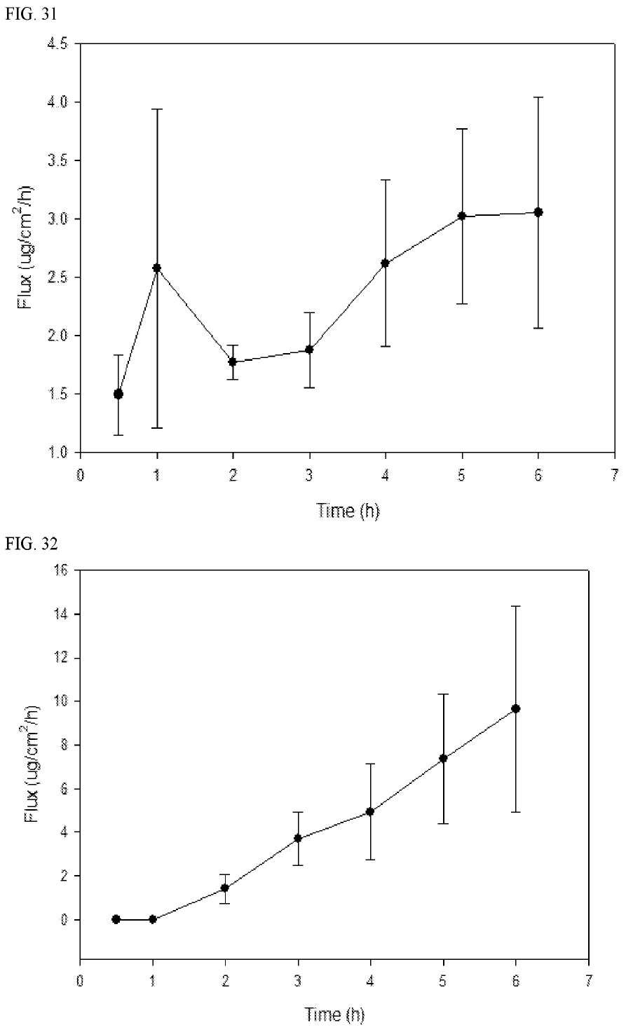

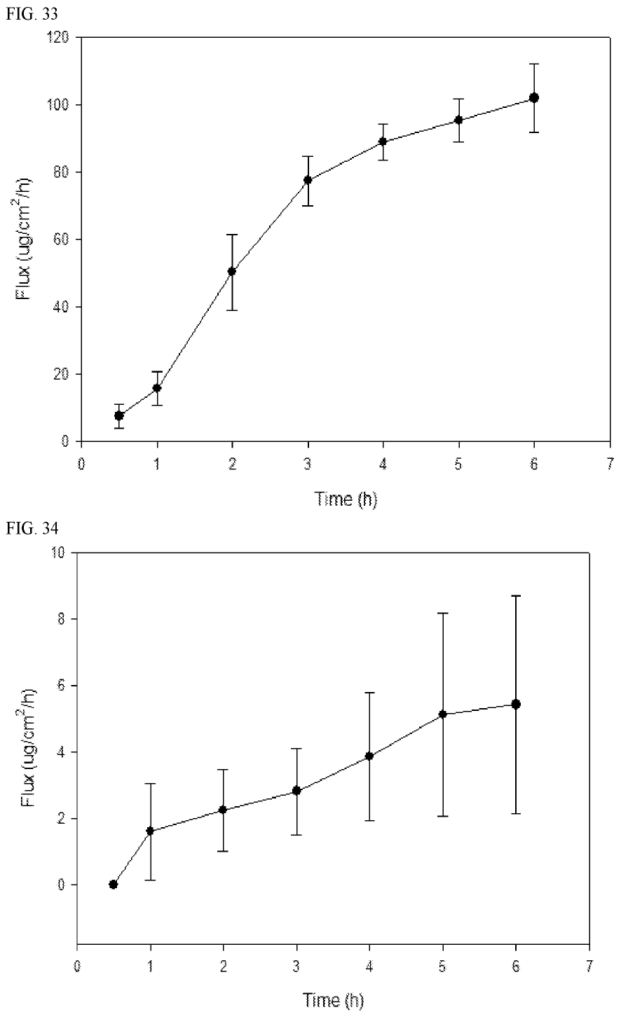

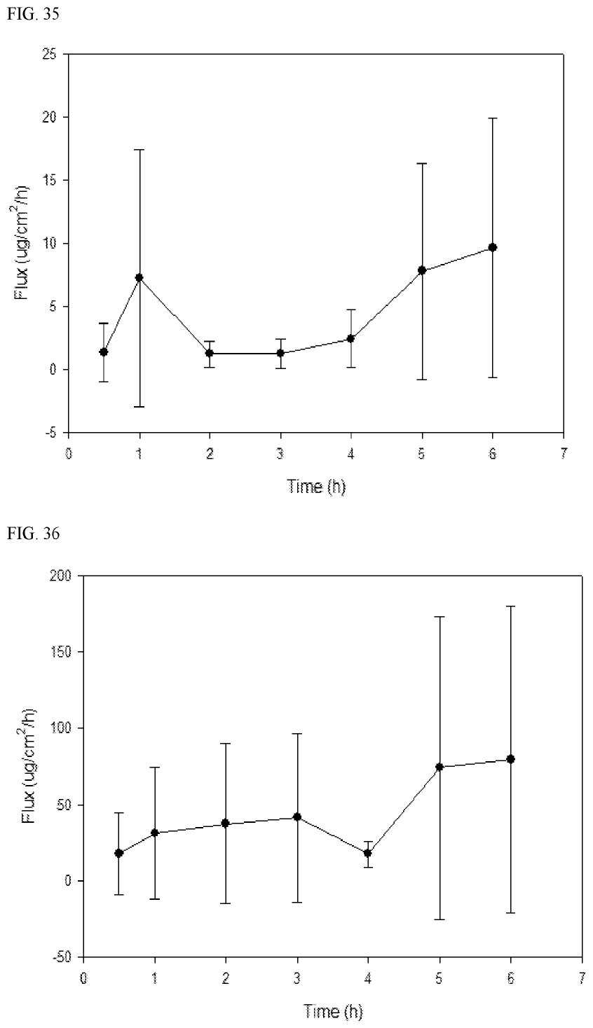

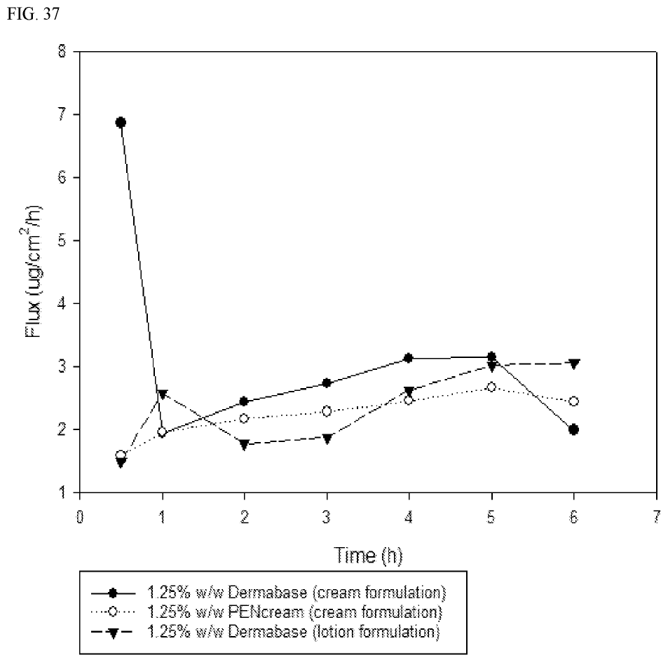

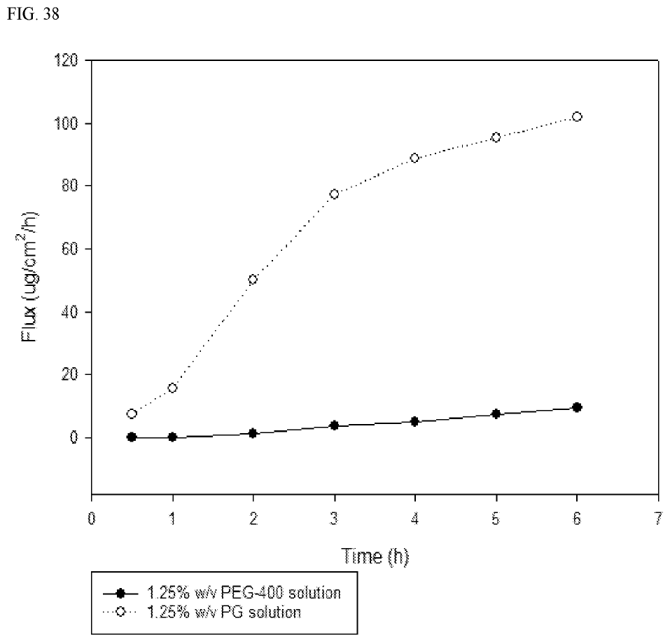

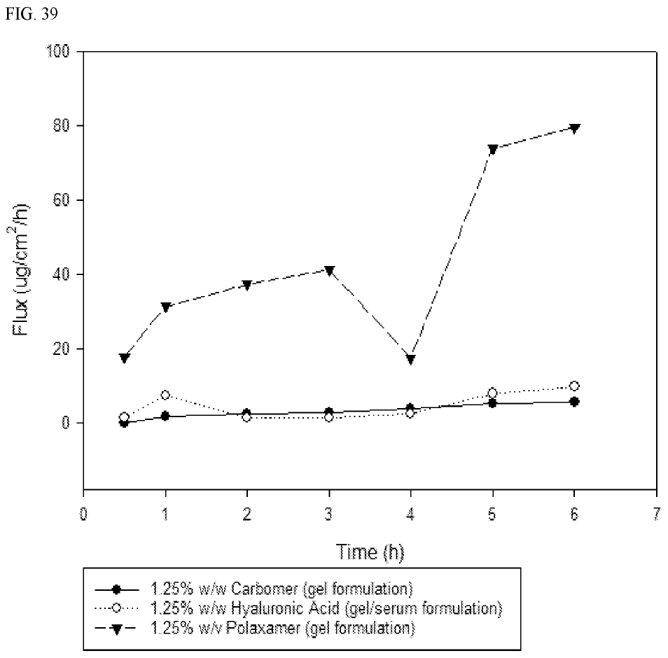

In vitro transdermal diffusion and permeation evaluation was performed using Strat-M® transdermal diffusion membrane as a synthetic non-animal-based model for transdermal diffusion in human skin. The analysis was conducted on the formulations of TAK-242 at 1.25%, 2.5% and 5% listed under the methods section in preparation of topical formulations by non-sterile compounding. From the 8 formulations the free-drug in PEG-400 solution at all concentrations and Carbomer gel at lowest concentration were the only exhibiting lag time, which was calculated from the linear equation (F(x)=0) at the steady state of the drug permeated profile. The lag time for PEG-400 solution was 1.24±0.34 at 1.25% w/v, 0.83±0.15 at 2.5% w/v and 2.4±0.29 at 5% w/v. The 1.25% (w/w) Carbomer showed 0.15±0.07. The Strat-M® permeation flux (μg/cm²/h), Lag time (h), and drug retention in the membrane (μg) parameters are shown in Table 9 and FIGS. 11-39 illustrate Flux versus time. The diffuses through the membrane in all formulation, although higher drug amount was found in the membrane.

The mass flux (J) values for the TAK-242 formulations were lower when PENcream was used, and concentration effect on the permeation profile of the drug increased significantly in proportion to the drug in the formulation. Additionally, in solution, it was observed that the type of solvent used had an effect in the permeation, allowing the discrimination between the effect of the solvent and the type of vehicle used.

Significant difference on flux was observed between PEG400 and PG solutions. In this study, the highest values of flux were obtained when HD formulations were used compared to the oil cream base used for 1.25 and 2.5 w/w formulations.

The flux values for Poloxamer HD at 2.5% was 14.5±5.92 μg/cm²/h in contrast to cream formulation with 6.64±2.05, 4.60±1.70, 1.91±1.14 for DermaBASE (cream), DermaBASE (lotion), and PENcream respectively, at the same concentration. This behavior was due to the hydrophobic character of TAK242 (log P=1.54). The hydrophilic HD allowed greater permeation and higher flow of TAK242 in shorter time. For the oil-based creams, the opposite was observed due to greater affinity for the hydrophobic active (Blohm-Mangone K, Burkett N B, Tahsin S, et al. Pharmacological TLR4 Antagonism Using Topical Resatorvid Blocks Solar UV-Induced Skin Tumorigenesis in SKH-1 Mice. Cancer Prevention Research. 2018; 11(5):265-278).

On the other hand, then highest drug retention after diffusion test was at 5% drug solution in PG, for 2.5% formulations; TAK-242 HD-based had the higher drug retention with 1129.42±217.5, 431.31±227.3, and 832.3±42.7 for 2.5% w/w Carbomer, 2.5% w/w Hyaluronic acid, and 2.5% w/v Pluronic F-127, respectively. For the 1.25% formulation, 1.25% w/w DermaBASE had the highest amount of drug retention in Strat-M membranes.

### In Vitro Cell Dose-Response Assay in 2-D Cell Culture

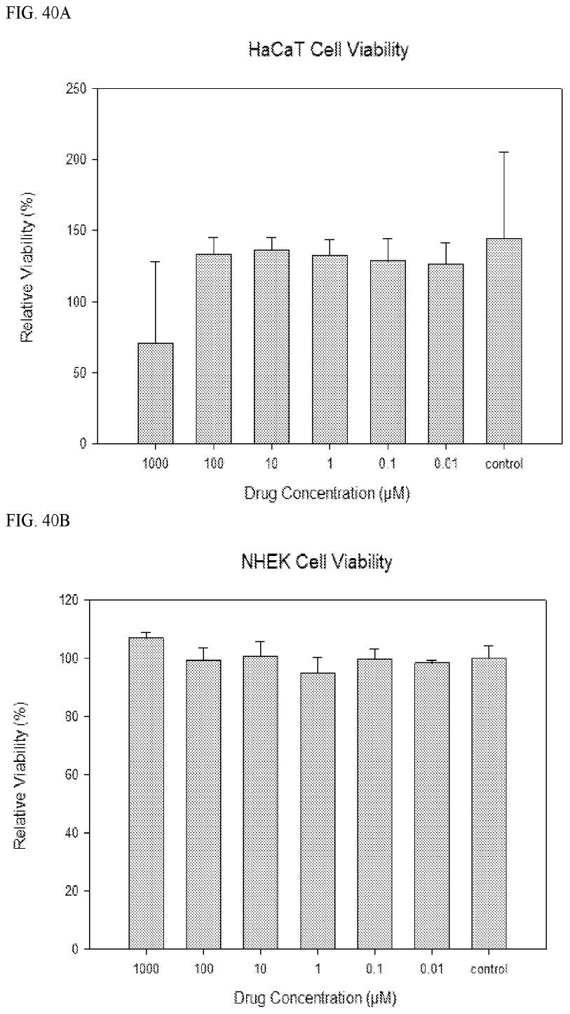

TAK-242 did not show any significant toxicity in either human transformed keratinocytes (HaCaT) as shown in FIG. 40A or primary normal epidermal keratinocytes (NHEK) as shown in FIG. 40B after 72 h exposure to a series of increasing concentrations of raw TAK-242. The viability of NHEK cells remained nearly 100% at all concentrations and viability of HaCat cells was close to 100% at all concentrations except at 1000 μM where % cell viability decreased to ˜70%.

In Vitro Permeation of Resatorvid (TAK-242) through 3-D Normal Human Derived Epidermal Keratinocytes (EpiDerm™)

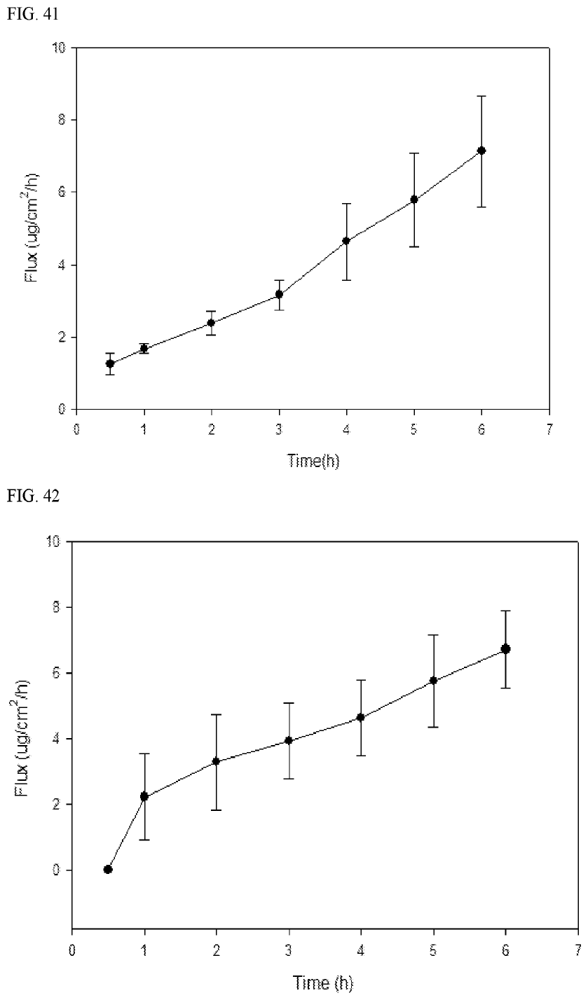

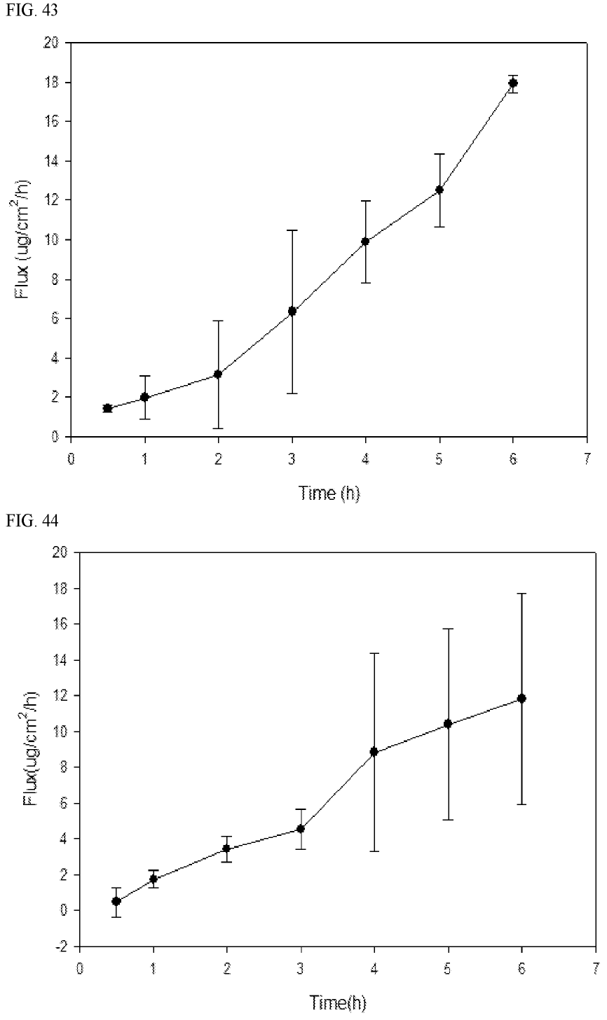

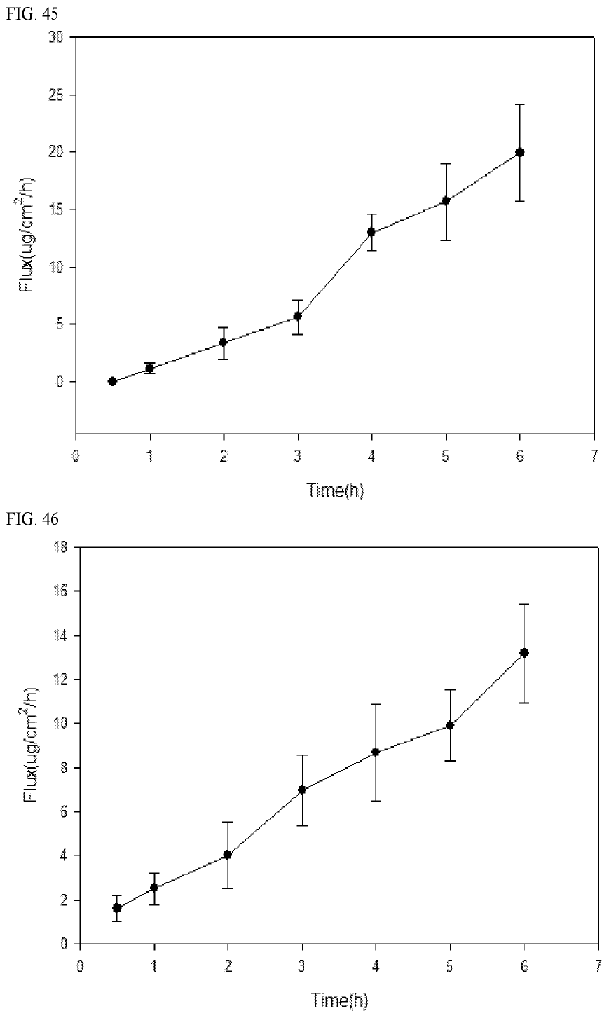

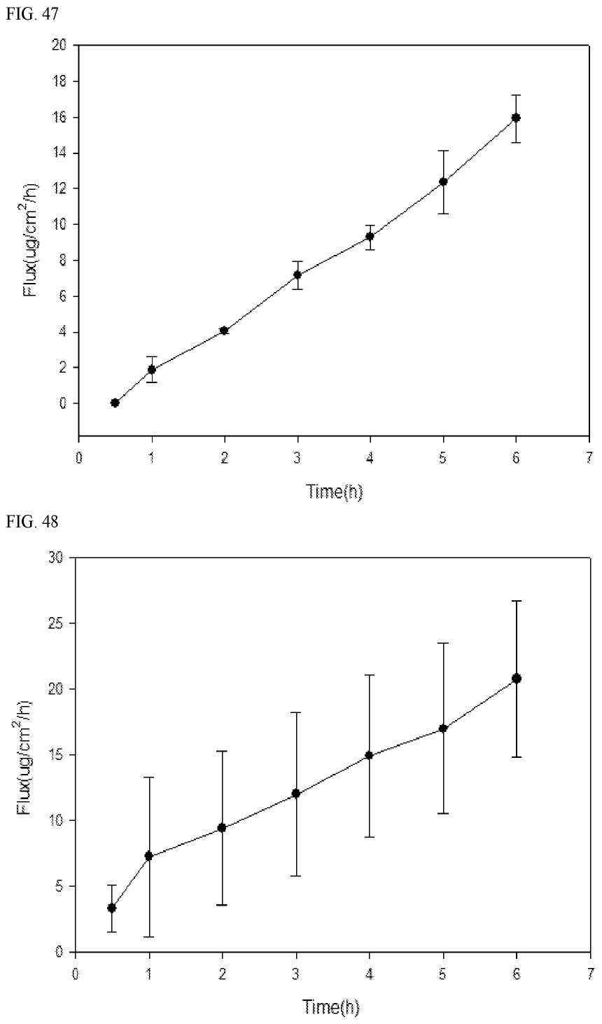

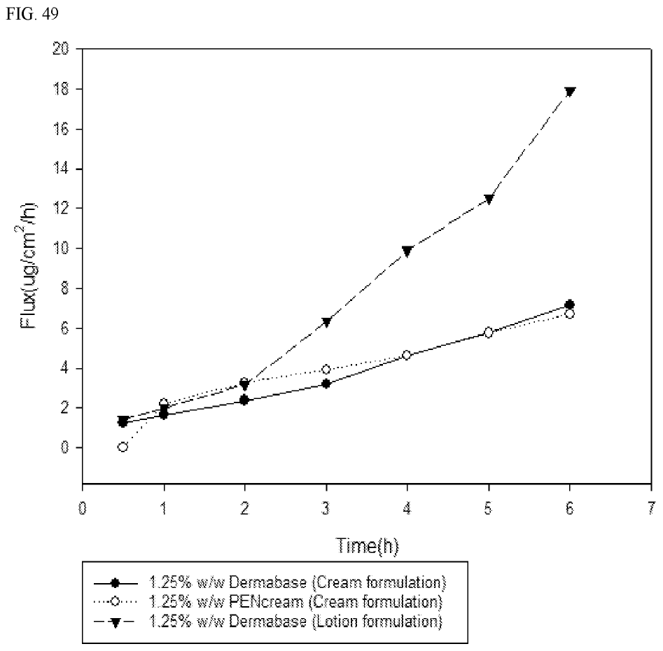

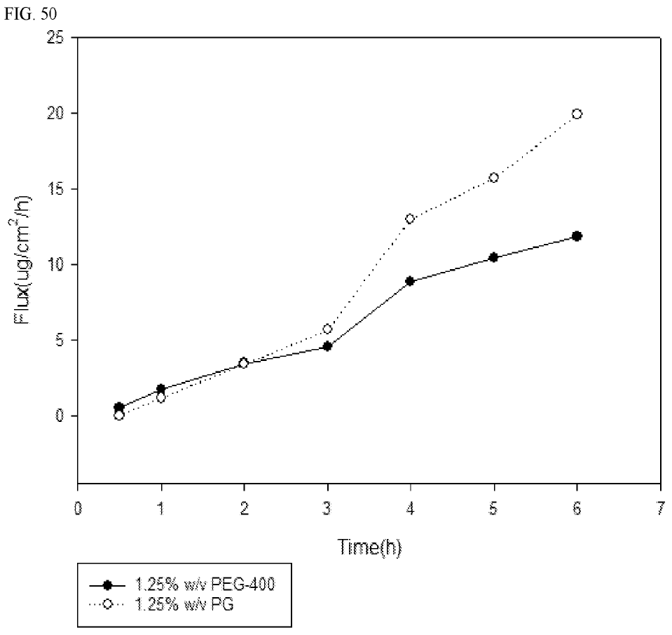

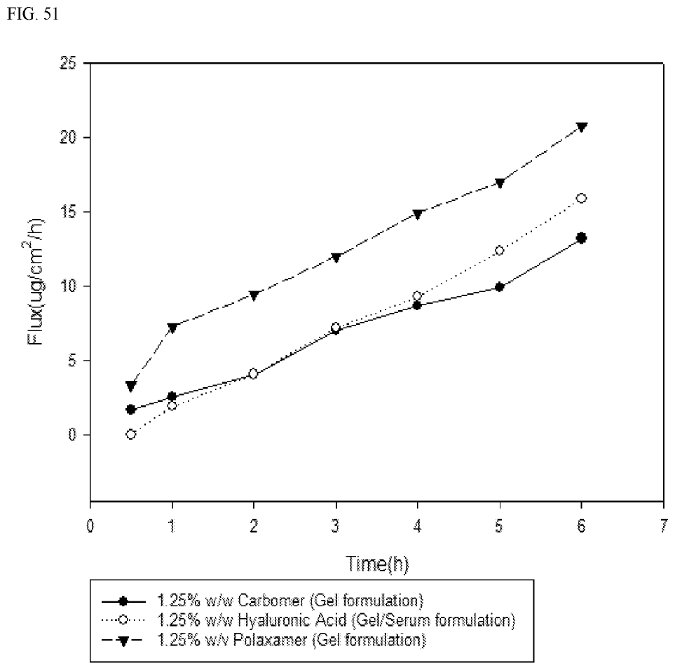

TAK-242 diffusion profiles from 1.25% topical formulations through an EpiDerm™ 3-D normal human derived epidermal keratocytes over 6 hours are presented in FIGS. 41-51. Flux values from 1.25% w/w DermaBASE were the highest followed by 1.25% w/w Hyaluronic Acid with 7.90±1.90 and 6.53±0.769, respectively. From the 8 formulations, the lotion DermaBASE, free-TAK-242 PEG and PG solution, and Hyaluronic acid HD showed lag time ≤0.5 h. For drug retention, Carbomer gel showed a higher value than free-TAK242 solutions, 51.06±12.4, 43.07±6.91and 21.35±6.54, respectively. For oil-based creams, PENcream showed highest drug retention with 19.69±2.16 followed by dermaBASE cream with 14.24±2.54. Diffusion through the 3-D cell model is also relevant as this predicts the TAK-242 penetration capacity in depth skin and accumulate in the tissue, which are essential for topical delivery of high molecular weight and hydrophobic molecules in carcinogenesis treatment.

In conclusion, eight topical formulations were successfully prepared, and their drug permeation was evaluated with two in-vitro models. In-vitro drug permeation studies were utilized to screen formulation candidates from different categories (solution, gel and cream/lotion). PG solution, carbomer gel and DermaBASE cream demonstrated better drug flux/permeation and retention in in-vitro models.

TABLE 6
DSC thermal analysis (n = 3, mean ± standard deviation)
Resatorvid (TAK-242)

| | Endotherm 1 | | Endotherm 2 | |
|---|---|---|---|---|
| | Tₚₑₐₖ | Enthalpy | Tₚₑₐₖ | Enthalpy |
| System | (° C.) | (J/g) | (° C.) | (J/g) |
| Raw | 52.5 ± 1.89 | 24.1 ± 3.31 | 69.3 ± 0.14 | 57.4 ± 2.71 |
| Resatorvid | | | | |

TABLE 7
Residual water content quantified by KFT. (n =
4, mean ± standard deviation)

| | Residual Water |
|---|---|
| Sample Identification | content (% w/w) |
| Resatorvid 1 | 0.074 |
| Resatorvid 2 | 0.090 |
| Resatorvid 3 | 0.052 |
| Resatorvid 4 | 0.069 |
| Average ± SD | 0.0173 ± 0.0156 |

TABLE 8
Summary of partition coefficient (Log-P) for Resatorvid (TAK-242)
at various pH. (n = 3, mean ± standard deviation)

| Experimental (Log P) | Average ± SD |
|---|---|
| At 35° C., pH = 6.5 | 1.65 ± 0.029 |
| At Room temperature/ambient temperature, pH = 6.5 | 1.58 ± 0.019 |

| Experimental (Log P) | Avg. Log-P ± SD |
|---|---|
| At 35° C., pH = 7.1 | 1.68 ± 0.013 |
| At Room temperature/ambient temperature, pH = 7.1 | 1.54 ± 0.149 |
| Experimental (Log P) | Avg. Log-P ± SD |
| At 35° C., pH = 8.8 | 0.94 ± 0.023 |
| At Room temperature/ambient temperature, pH = 8.8 | 0.88 ± 0.060 |

| Predicted (cLog P) | Predicted value |
|---|---|
| ChemDraw Version 16.0 | 2.53 |
| Swiss ADME | 3.04 |

TABLE 9
Skin permeation parameters of Resatorvid (TAK-242) topical formulations through
Strat-M ® transdermal diffusion membrane. (n = 3, mean ± standard deviation)

| | Flux | Lag | Drug |
|---|---|---|---|
| Formulation | (μg/cm²/h) | time (h) | retention (μg) |
| 5% w/w DermaBASE | 2.35 ± 1.20 | — | 274.05 ± 8.48 |
| (cream formulation) | | | |
| 5% w/w DermaBASE | 1.85 ± 0.93 | — | 266.27 ± 10.9 |
| (lotion formulation) | | | |
| 5% w/w PENcream | 1.45 ± 0.73 | — | 261.51 ± 20.9 |
| (cream formulation) | | | |
| 5% w/v PEG-400 solution | 3.17 ± 1.08 | 2.4 ± 0.29 | 231.18 ± 68.3 |
| 5% w/v PG solution | 5.60 ± 1.20 | — | 296.87 ± 14.9 |
| 2.5% w/w DermaBASE | 6.64 ± 2.05 | — | 301.36 ± 16.3 |
| (cream formulation) | | | |
| 2.5% w/w DermaBASE | 4.60 ± 1.70 | — | 107.37 ± 7.63 |
| (lotion formulation) | | | |
| 2.5% w/w PENcream | 1.91 ± 1.14 | — | 95.61 ± 0.40 |
| (cream formulation) | | | |
| 2.5% w/v PEG-400 solution | 4.39 ± 2.34 | 0.83 ± 0.15 | 102.8 ± 21.7 |
| 2.5% w/v PG solution | 17.5 ± 1.52 | — | 380.42 ± 64.7 |
| 2.5% w/w Carbomer | 14.5 ± 5.92 | — | 1129.42 ± 217.5 |
| (gel formulation) | | | |
| 2.5% w/w Hyaluronic Acid | 8.90 ± 2.10 | — | 431.31 ± 227.3 |
| (gel/serum formulation) | | | |
| 2.5% w/v Pluronic F-127 | 13.2 ± 7.20 | — | 832.3 ± 42.7 |
| (gel formulation) | | | |
| 1.25% w/w DermaBASE | 0.78 ± 0.20 | — | 177.21 ± 6.34 |
| (cream formulation) | | | |
| 1.25% w/w DermaBASE | 0.579 ± 0.132 | — | 628.19 ± 37.9 |
| (lotion formulation) | | | |
| 1.25% w/w PENcream | 0.313 ± 0.09 | — | 284.56 ± 79.4 |
| (cream formulation) | | | |
| 1.25% w/v PEG-400 solution | 1.91 ± 0.90 | 1.14 ± 0.34 | 269.24 ± 20.2 |
| 1.25% w/v PG solution | 26.9 ± 2.64 | — | 313 ± 80.7 |
| 1.25% w/w Carbomer | 1.01 ± 0.45 | 0.15 ± 0.07 | 298.04 ± 68.5 |
| (gel formulation) | | | |
| 1.25% w/w Hyaluronic Acid | 2.30 ± 4.10 | — | 250 ± 31.4 |
| (gel/serum formulation) | | | |
| 1.25% w/v Pluronic F-127 | 8.45 ± 10.51 | — | 197.82 ± 63.9 |
| (gel formulation) | | | |

TABLE 10
Skin permeation parameters of Resatorvid (TAK-242) topical formulations
through EpiDerm ™ 3-D normal human derived epidermal keratinocytes
as models for transdermal diffusion. (n = 3, mean ± standard deviation)

| | Flux | Lag | Drug |
|---|---|---|---|
| Formulation | (μg/cm²/h) | time (h) | retention (μg) |
| 1.25% w/w DermaBASE | 2.56 ± 0.68 | — | 14.24 ± 2.54 |
| (cream formulation) | | | |
| 1.25% w/w DermaBASE | 7.90 ± 1.90 | 0.20 ± 0.08 | 9.38 ± 2.56 |
| (lotion formulation) | | | |
| 1.25% w/w PENcream | 1.97 ± 0.38 | — | 19.69 ± 2.16 |
| (cream formulation) | | | |
| 1.25% w/v PEG-400 | 2.39 ± 0.68 | 0.37 ± 0.11 | 21.35 ± 6.54 |
| solution | | | |
| 1.25% w/v PG solution | 11.08 ± 2.92 | 0.50 ± 0.16 | 43.07 ± 6.91 |
| 1.25% w/w Carbomer | 5.34 ± 1.27 | — | 51.06 ± 12.4 |
| (gel formulation) | | | |
| 1.25% w/w Hyaluronic Acid | 6.53 ± 0.769 | 0.36 ± 0.0.18 | 24.02 ± 6.63 |
| (gel/serum formulation) | | | |
| 1.25% w/v Pluronic F-127 | 6.37 ± 0.47 | — | 18.52 ± 2.08 |
| (gel formulation) | | | |

TABLE 11
in vitro stability

| Expt with the | PG Soln | Carbomer Gel | Dermabase Lotion | Dermabase Cream |
|---|---|---|---|---|
| Formulations | (Vehicle 1) | (Vehicle 2) | (Vehicle 3) | (Vehicle 4) |
| 1-Month | Physical/Acceptable | Physical/Acceptable | Physical/Acceptable | Physical/Acceptable |
| Physical and | Chemical/Acceptable | Chemical/Acceptable | Chemical/Acceptable | Chemical/Acceptable |
| Chemical | | | | |
| Stability | | | | |
| 3- Month | Physical/Acceptable | Physical/Acceptable | Physical/Acceptable | Physical/Acceptable |
| Physical and | Chemical/Acceptable | Chemical/Acceptable | Chemical/Acceptable | Chemical/Acceptable |
| Chemical | | | | |
| Stability | | | | |
| 6- Month | Physical/Acceptable | Physical/NOT | Physical/NOT | Physical/NOT |
| Physical and | | Acceptable | Acceptable | Acceptable |
| Chemical | | Condition at 40° C., | Condition at 40° C., | Condition at 40° C., |
| Stability | | 75% RH | 75% RH | 75% RH |
| | Chemical/Acceptable | Chemical/Acceptable | Chemical/Acceptable | Chemical/Acceptable |

Stability studies were conducted under the following 4 sets of conditions:
4° C./ambient RH
25° C./ambient RH
25° C./60% RH
40° C./75% RH
All formulations exhibited acceptable stability.

Example 4

### In Vivo Analysis

Acute in vivo analysis of Resatorvid in mice was performed. FIG. 52 shows the experimental protocol. Mice were topically treated with Resatorvid before and/or after solar simulated light (SSL). Skin is harvested for analysis 24 hours after SSL.

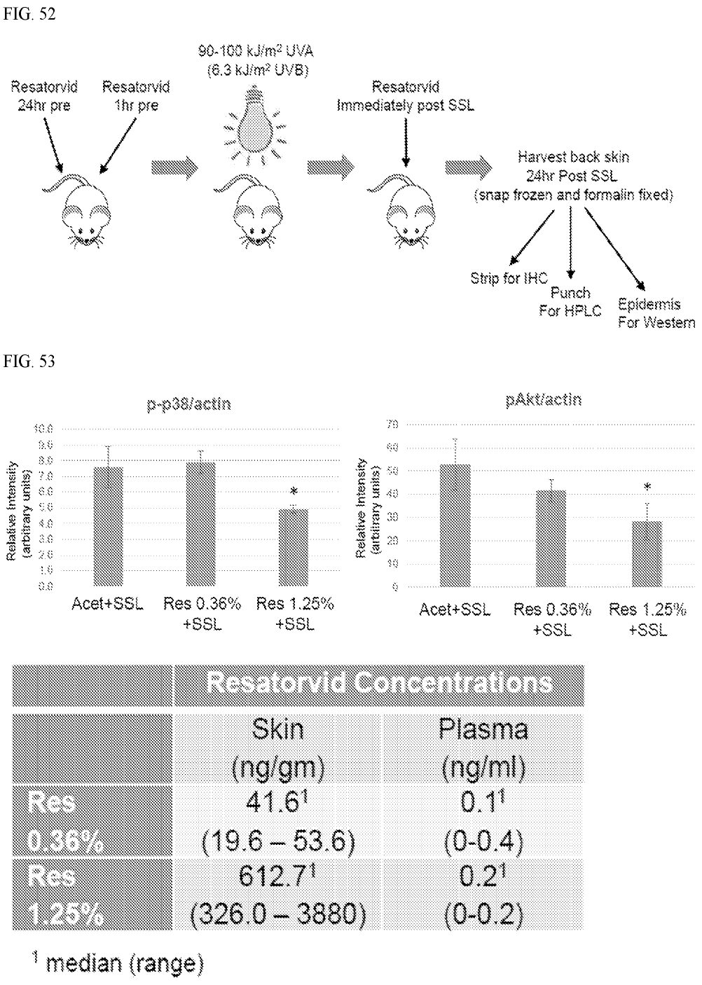

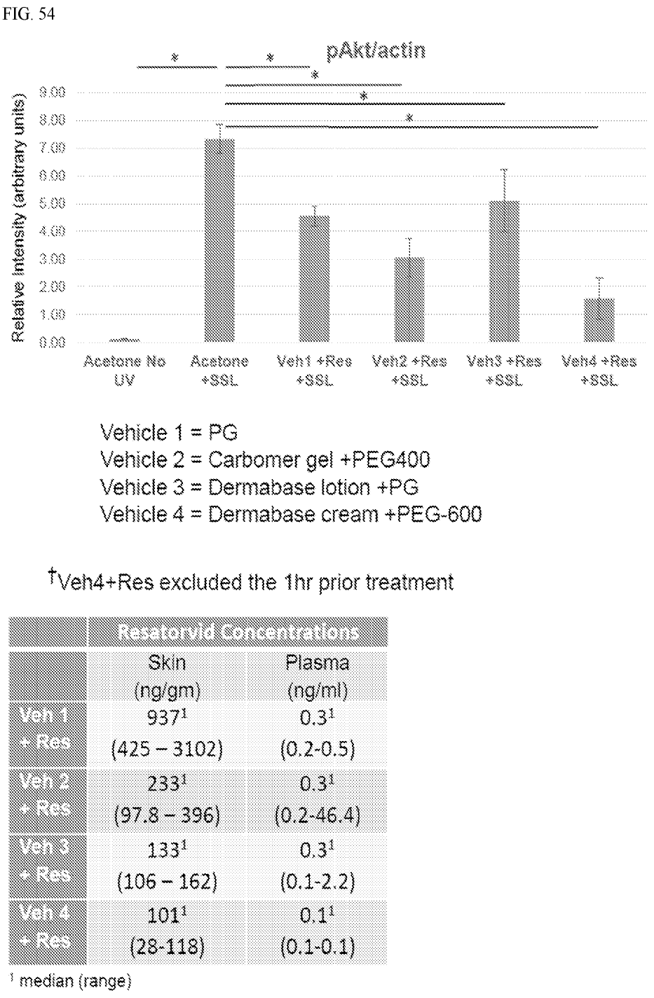

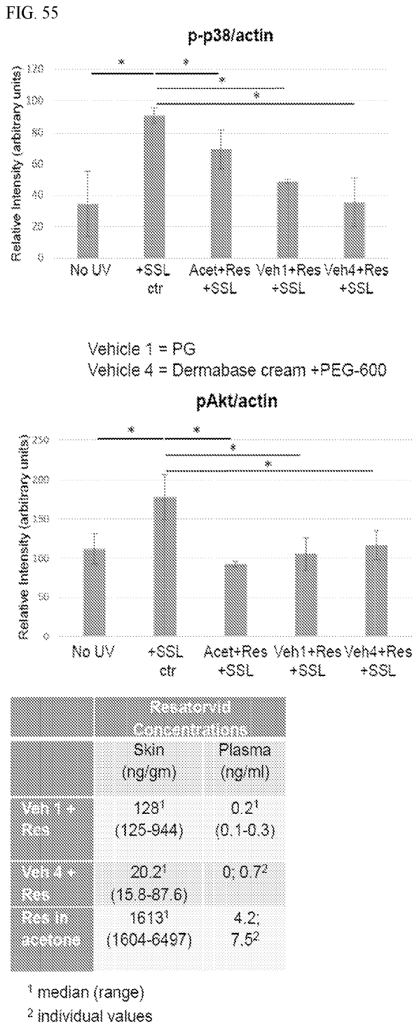

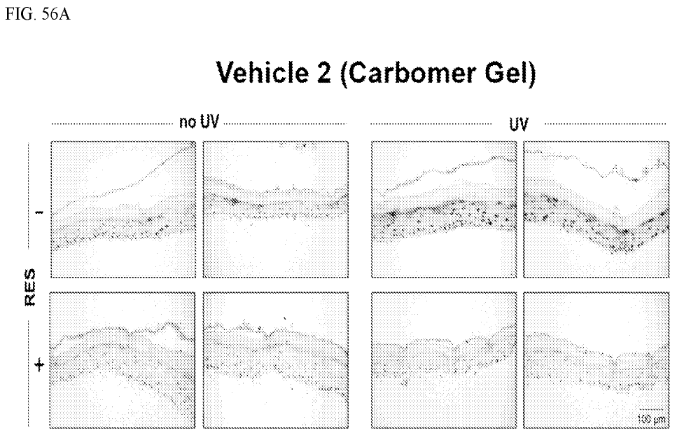

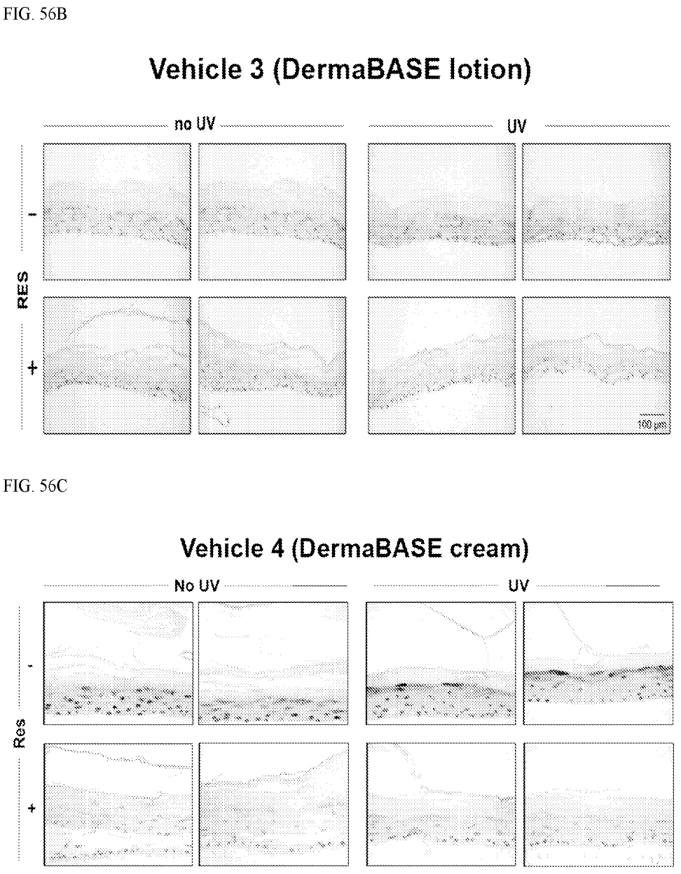

Results are shown in FIGS. 53-56. Topical resatorvid (RES) potently blocked p-p38 signal in EpiDerms (constitutive or UV-induced); this is attributable to compound and not vehicle.

p-p38 signal was detectable in vehicle only treated skin skin; UV -induced p-p38 signal is moderately elevated.

Vehicle 2, 3 and 4 were well tolerated by the EpiDerm reconstructs.

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

We claim:

1. A composition, comprising:
a pharmaceutical composition comprising resatorvid and a pharmaceutically acceptable carrier, wherein said composition is formulated for topical delivery.

2. The composition of claim 1, wherein said composition has a dosage form selected from the group consisting of a cream, a liquid, a solution, a hydrogel, and a lotion.

3. The composition of claim 2, wherein said cream and/or lotion comprises an emollient.

4. The composition of claim 3, wherein said emollient comprises one or more of petrolatum, lanolin, mineral oil, dimethicone, glycerin, lecithin, and propylene glycol.

5. The composition of claim 3, wherein said cream further comprises polyethylene glycol (PEG) 400 USP, PEG 600 USP, or PG USP.

6. The composition of claim 4 or 5, wherein said PEG is present at a concentration of 0.1 to 2 ml per 1 gram of cream.

7. The composition of claim 2, wherein said liquid comprises PEG USP or PG USP.

8. The composition of claim 7, wherein said PEG is PEG 400 USP or PEG 600 USP.

9. The composition of claim 2, wherein said hydrogel comprises poly(acrylic acid), a poloxamer, and/or hyaluronate.

10. The composition of any of the preceding claims, wherein said composition is part of a skin patch or microneedle skin patch.

11. The composition of any of the preceding claims, wherein said resatorvid is a racemic mixture or an enantiomer.

12. The composition of any of the preceding claims, wherein said resatorvid is present in said pharmaceutical composition at a concentration of 0.01 to 10% w/v or w/w.

13. The composition of claim 12, wherein said resatorvid is present in said pharmaceutical composition at a concentration of 0.1 to 5.0% w/v or w/w.

14. The composition any of the preceding claims, wherein said pharmaceutical composition is hydrophobic.

15. The composition of any of the preceding claims, wherein said resatorvid inhibits TLR4.

16. A method of treating or preventing a skin pathology, comprising:
topically administering the composition of any one of claims 1 to 15 to a subject in need thereof.

17. The method of claim 16, wherein said skin pathology is selected from the group consisting of pathologies related to exposure to environmental stressors, photoaging and chronological aging, inflammatory dysregulation, microbial infection, and skin malignancies and premalignancies.

18. The method of claim 17, wherein said environmental stressor is selected from the group consisting of solar radiation, ionizing radiation, pollutants, and natural and artificial allergens.

19. The method of claim 18, wherein said solar radiation is selected from the group consisting of ultraviolet, visible, and infrared.

20. The method of claim 18, wherein said pollutants are selected from the group consisting of dioxin, benzpyrene, particulate matter, and arsenic.

21. The method of claim 17, wherein said inflammatory dysregulation is selected from the group consisting of erythema, urticaria, telangiectasia, edema, impaired skin barrier function, atopic dermatitis, contact dermatitis, eczematous dermatitis, allergic reactions, autoimmune conditions, scleroderma, and wounds.

22. The method of claim 21, wherein said autoimmune condition is lupus or psoriasis.

23. The method of claim 21, wherein said wound is a diabetic wound, a burn wound, or a radiation-induced injury wound.

24. The method of claim 17, wherein said microbial infection is a fungal, bacterial, or viral pathogen.

25. The method of claim 17, wherein said skin malignancies and premalignancies are selected from the group consisting of melanoma, nonmelanoma skin cancer, actinic keratosis, and dysplastic nevi.

26. A method of inhibiting TLR4 in the skin of a subject, comprising:
topically administering the composition of any one of claims 1 to 15 to a subject in need thereof.

27. The method of claim 26, wherein said inhibiting treats or prevents a skin pathology.

28. The use of the composition of any one of claims 1 to 15 to treat or prevent a skin pathology in a subject.

29. The composition of any one of claims 1 to 15 for use in treating or preventing a skin pathology in a subject.

30. The use of the composition of any one of claims 1 to 15 to treat or prevent a skin pathology in a subject.

31. The composition of any one of claims 1 to 13 for use in treating or preventing a skin pathology in a subject.

32. A method of preparing the composition of any one of claims 1 to 13, comprising:
a) dissolving resatorvid in a liquid selected from the group consisting of PEG-600 and PEG-400 using sonication to generate a resatorvid solution; and
b) mixing said resatorvid solution and said pharmaceutically acceptable carrier to generate said composition.

33. The composition of any one of claims 1 to 13, wherein said composition is made by a method, comprising:
a) dissolving resatorvid in a liquid selected from the group consisting of PEG-600 and PEG-400 using sonication to generate a resatorvid solution; and
b) mixing said resatorvid solution and said pharmaceutically acceptable carrier to generate said composition.
